SEQUENCE LISTING (29 sequences)

SEQ ID NO: 1 - protein, 107 aa, Artificial Sequence
  Synthetic peptide
DILMTQSPSSLSASPGDRVTISCRASQDIRNNLNWYQQKPGQAPRLLIYYSSNLHSGVPDRFSGSGSGTDFTLTISRLEPEDFAAYYCQQSIKLPLTFGSGTKVEIK

SEQ ID NO: 2 - protein, 107 aa, Artificial Sequence
  Synthetic peptide
EIELTQSPSSLSVSLGDRVTISCSASQDIRNNLNWYQQKPGQAPRLLIYYSSNLQTGVPARFSGSGSGTDFTLTISRLEPEDVAAYYCQQSIKLPFTFGSGTKVEIK

SEQ ID NO: 3 - protein, 107 aa, Artificial Sequence
  Synthetic peptide
DIEMTQSPSSLSASAGERVTISCRASQGIRNSLNWYQQKPGQAPRLLIYYSSNLQTGVPSRFSGSGSGTDFTLTISRLQAEDFATYYCQQSNKLPFTFGSGTKVEIK

SEQ ID NO: 4 - protein, 107 aa, Artificial Sequence
  Synthetic Peptide
DILLTQSPSSLSATPGERATITCRASQGIRNNLNWYQQKPGQAPRLLIYYSSNLQSGVPSRFSGSGSGTDFTLTISRLQPEDVAAYYCQQSIKLPFTFGSGTKVEIK

SEQ ID NO: 5 - protein, 107 aa, Artificial Sequence
  Synthetic peptide
EIEMTQSPSSLSVSAGERATISCSASQDIRNSLNWYQQKPGQAPRLLIYYSSNLHTGVPARFSGSGSGTDFTLTISRLEPEDVAIYYCQQSNKLPLTFGSGTKVEIK

SEQ ID NO: 6 - protein, 107 aa, Artificial Sequence
  Syntetic peptide
EIELTQSPSSLSVSPGDRVTISCSASQGIRNSLNWYQQKPGQAPRLLIYYSSNLHTGVPARFSGSGSGTDFTLTISRLQAEDFATYYCQQSIKLPLTFGSGTKVEIK

SEQ ID NO: 7 - protein, 107 aa, Artificial Sequence
  Synthetic peptide
DILMTQSPSSLSASPGDRVTISCRASQDIRNNLNWYQQKPGQAPRLLIYYSSNLQTGVPARFSGSGSGTDFTLTISRLEPEDFAAYYCQQSIKLPLTFGSGTKVEIK

SEQ ID NO: 8 - protein, 116 aa, Artificial Sequence
  Synthetic peptide
EVQLVESGAGVKQPGGTLRLTCTASGFSLTTYGVHWVRQAPGKGLEWVGVIWGDGRTDYDAAFMSRVTISKDTSKSTVYLQMNSLRAEDTAVYYCMRNRHDWFDYWGQGTTVTVSS

SEQ ID NO: 9 - protein, 116 aa, Artificial Sequence
  Synthetic peptide
EVQLVQSGGGVKQPGETLRLTCTASGFSLTTYGVHWVRQAPGKGLEWVGVIWGDGRTDYDAAFMSRVTISKDTSKSTAYLQMNSLRAEDTAVYYCMRNRHDWFDYWGQGTTVTVSS

SEQ ID NO: 10 - protein, 116 aa, Artificial Sequence
  Synthetic peptide
EVQLVESGAGVEQPGGTLRLTCTASGFSLTTYGVHWVRQAPGKGLEWMGVIWGDGRTDYDAAFMSRVTISRDTSKSTAYLQLNSLRAEDTAVYYCVRNRHDWFDYWGQGTTVTVSS

SEQ ID NO: 11 - protein, 116 aa, Artificial Sequence
  Synthetic peptide
EVQLVESGAELVQPGGSLRLTCKASGFTLNTYGVHWVRQAPGKGLEWMGVIWGGGRTDYDASFMSRVTISKDNSKNTAYLQLNSLRAEDTAVYYCTRSRHDWFDYWGQGTTVTVSS

SEQ ID NO: 12 - protein, 116 aa, Artificial Sequence
  Synthetic peptide
EVQLVQSGGGLKQPGETLRLSCTASGYSLTTYGVHWVRQAPGKGLEWMGVIWGDGRTDYDSSFMSRVTISKDTSKSTAYLQLNSLRAEDTAVYYCTRNRHDWFDYWGQGTTVTVSS

SEQ ID NO: 13 - protein, 116 aa, Artificial Sequence
  Synthetic peptide
EVQLVQSGGGVKQPGETLRLTCTASGFSLSTYGVHWVRQAPGKGLEWVGVIWGDGRTDYDAAFMSRVTISKDTSKSTVYLQMNSLRAEDTAVYYCMRNRHDWFDYWGQGTTVTVSS

SEQ ID NO: 14 - protein, 116 aa, Artificial Sequence
  Synthetic peptide
EVQLVESGGGVKQPGETLRLTCTASGFSLSTYGVHWVRQAPGKGLEWVGVIWGDGRTDYDAAFMSRVTISKDTSKSTVYLQMNSLRAEDTAVYYCMRNRHDWFDYWGQGTTVTVSS

SEQ ID NO: 15 - protein, 116 aa, Artificial Sequence
  Synthetic peptide
  MISC_FEATURE (6)..(6): Xaa is Glu or Gln
  MISC_FEATURE (9)..(9): Xaa is Ala or Gly
  MISC_FEATURE (10)..(10): Xaa is Gly or Glu
  MISC_FEATURE (11)..(11): Xaa is Leu or Val
  MISC_FEATURE (12)..(12): Xaa is Val, Lys or Glu
  MISC_FEATURE (16)..(16): Xaa is Gly or Glu
  MISC_FEATURE (17)..(17): Xaa is Thr or Ser
  MISC_FEATURE (21)..(21): Xaa is Thr or Ser
  MISC_FEATURE (23)..(23): Xaa is Thr or Lys
  MISC_FEATURE (27)..(27): Xaa is Phe or Tyr
  MISC_FEATURE (28)..(28): Xaa is Ser or Thr
  MISC_FEATURE (30)..(30): Xaa is Thr, Asn or Ser
  MISC_FEATURE (48)..(48): Xaa is Val or Met
  MISC_FEATURE (54)..(54): Xaa is Gly or Asp
  MISC_FEATURE (61)..(61): Xaa is Ala or Ser
  MISC_FEATURE (62)..(62): Xaa is Ala or Ser
  MISC_FEATURE (71)..(71): Xaa is Lys or Arg
  MISC_FEATURE (73)..(73): Xaa is Asn or Thr
  MISC_FEATURE (76)..(76): Xaa is Ser or Asn
  MISC_FEATURE (78)..(78): Xaa is Val or Ala
  MISC_FEATURE (82)..(82): Xaa is Met or Leu
  MISC_FEATURE (96)..(96): Xaa is Val, Met or Thr
  misc_feature (98)..(98): Xaa can be any naturally occurring amino acid
EVQLVXSGXXXXQPGXXARLXCXASGXXLXTYGVHWVRQAPGKGLEWXGVIWGXGRTDYDXXFMSRVTISXDXSKXTXYLQXNSLRAEDTAVYYCXRXRHDWFDYWGQGTTVTVSS

SEQ ID NO: 16 - protein, 107 aa, Artificial Sequence
  Synthetic peptide
  MISC_FEATURE (1)..(1): Xaa is Asp or Glu
  MISC_FEATURE (3)..(3): Xaa is Leu or Glu
  MISC_FEATURE (4)..(4): Xaa is Met or Leu
  MISC_FEATURE (13)..(13): Xaa is Ala or Val
  MISC_FEATURE (14)..(14): Xaa is Ser or Thr
  MISC_FEATURE (15)..(15): Xaa is Leu, Pro or Ala
  MISC_FEATURE (17)..(17): Xaa is Asp or Glu
  MISC_FEATURE (19)..(19): Xaa is Val or Ala
  MISC_FEATURE (22)..(22): Xaa is Thr or Ser
  MISC_FEATURE (24)..(24): Xaa is Ser or Arg
  MISC_FEATURE (28)..(28): Xaa is Gly or Asp
  MISC_FEATURE (32)..(32): Xaa is Ser or Asn
  MISC_FEATURE (55)..(55): Xaa is His or Gln
  MISC_FEATURE (56)..(56): Xaa is Ser or Thr
  MISC_FEATURE (60)..(60): Xaa is Ser, Asp or Ala
  MISC_FEATURE (79)..(79): Xaa is Glu or Gln
  MISC_FEATURE (80)..(80): Xaa is Pro or Ala
  MISC_FEATURE (83)..(83): Xaa is Phe or Val
  MISC_FEATURE (85)..(85): Xaa is Thr, Ala or Ile
  MISC_FEATURE (92)..(92): Xaa is Ile or Asn
  MISC_FEATURE (96)..(96): Xaa is Phe or Leu
XIXXTQSPSSLSXXXGXRXTIXCXASQXIRNXLNWYQQKPGQAPRLLIYYSSNLXXGVPXRFSGSGSGTDFTLTISRLXXEDXAXYYCQQSXKLPXTFGSGTKVEIK

SEQ ID NO: 17 - protein, 465 aa, Artificial Sequence
  Synthetic pepitde
MEWSWVFLFFLSVTTGVHSEVQLVESGAGVKQPGGTLRLTCTASGFSLTTYGVHWVRQAPGKGLEWVGVIWGDGRTDYDAAFMSRVTISKDTSKSTVYLQMNSLRAEDTAVYYCMRNRHDWFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 234 aa, Artificial Sequence
  Synthetic peptide
MSVPTQVLGLLLLWLTDARCDILLTQSPSSLSATPGERATITCRASQGIRNNLNWYQQKPGQAPRLLIYYSSNLQSGVPSRFSGSGSGTDFTLTISRLQPEDVAAYYCQQSIKLPFTFGSGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 13 aa, Artificial Sequence
  Synthetic peptide
YSLRWISDHEYLY

SEQ ID NO: 20 - protein, 13 aa, Artificial Sequence
  Synthetic peptide
LEYNYVKQWRHSY

SEQ ID NO: 21 - protein, 13 aa, Artificial Sequence
  Synthetic peptide
TWSPVGHKLAYVW

SEQ ID NO: 22 - protein, 13 aa, Artificial Sequence
  Synthetic peptide
LWWSPNGTFLAYA

SEQ ID NO: 23 - protein, 13 aa, Artificial Sequence
  Synthetic peptide
RISLQWLRRIQNY

SEQ ID NO: 24 - protein, 13 aa, Artificial Sequence
  Synthetic peptide
YVKQWRHSYTASY

SEQ ID NO: 25 - protein, 13 aa, Artificial Sequence
  Synthetic peptide
EEEVFSAYSALWW

SEQ ID NO: 26 - protein, 13 aa, Artificial Sequence
  Synthetic peptide
DYSISPDGQFILL

SEQ ID NO: 27 - protein, 13 aa, Artificial Sequence
  Synthetic peptide
SISPDGQFILLEY

SEQ ID NO: 28 - protein, 13 aa, Artificial Sequence
  Synthetic peptide
IYVKIEPNLPSYR

SEQ ID NO: 29 - protein, 15 aa, Artificial Sequence
  Synthetic peptide
GGGGSGGGGSGGGGS

(19) United States
(12) Patent Application Publication
(10) Pub. No.: US 2025/0270346 A1
(43) Pub. Date: Aug. 28, 2025

(54) EFFICACIOUS ANTI-CD26 ANTIBODY BIOMARKER

(71) Applicant: Y'S AC CO., LTD., Tokyo (JP)

(72) Inventors: Chikao Morimoto, Tokyo (JP); Naoto Hirota, Tokyo (JP); Ryo Hatano, Yokohama-shi (JP); Eric Angevin, Bures sur Yvette (FR); Yutaro Kaneko, Tokyo (JP); Fanny Valleix, Tokyo (JP); Nam Dang, Tokyo (JP)

(21) Appl. No.: 18/551,984

(22) PCT Filed: Mar. 22, 2022
(86) PCT No.: PCT/JP2022/013309

§ 371 Date: May 30, 2024

(30) Foreign Application Priority Data
Mar. 22, 2021 (JP) 2021-047954

(51) Int. Cl. C07K 16/40; C07K 16/28

(52) U.S. Cl. CPC C07K 16/40; C07K 16/2896; C07K 2317/24; C07K 2317/76

(57) ABSTRACT

Potential prognostic biomarkers for CD26-targeted therapy were identified based on phase I study data of a humanized anti-CD26 monoclonal antibody YS110 against CD26-expressing tumors. Using boxplot analysis, scatter plot analysis, Pearson's product-moment correlation/Spearman's rank-difference correlation, bar graph analysis, and receiver operating characteristic (ROC), a correlation between soluble CD26 titer variation and tumor volume variation by YS110 administration, RECIST criteria evaluation, and progression-free survival (PFS) were examined. Further, mechanism of serum soluble CD26 titer variation was confirmed by in vitro experiments. As a result, serum soluble CD26/DPP4 titer variation at an early stage of YS110 treatment was found for the first time as a predictive biomarker to evaluate a therapeutic effect.

### CROSS-REFERENCE TO RELATED APPLICATIONS

This international application claims priority of Japanese Patent Application No. 2021-047954 filed with the Japanese Patent Office on Mar. 22, 2021, and the entire contents of Japanese Patent Application No. 2021-047954 are incorporated herein by reference.

### TECHNICAL FIELD

The present invention relates to a biomarker for determining efficacy of an anti-CD26 antibody.

### BACKGROUND ART

CD26 is a 110 kDa type II transmembrane glycoprotein with dipeptidyl peptidase 4 (DPP4) activity in its extracellular domain, in which an N-terminal dipeptide can be cleaved at an anterior terminal position (Non Patent Literatures 1 and 2) with L-proline or L-alanine. CD26 has multiple biological functions and is expressed in various normal cell types and tumors. CD26 is also found as a soluble form with DPP4 activity stored in serum and other body fluids. In vitro and in vivo administration of an anti-CD26 monoclonal antibody blocks tumor growth, migration, and invasion through multiple mechanisms of action, extending a survival period of mouse xenogeneic transplant models inoculated with various cancers, including renal cell carcinoma (RCC) and malignant mesothelioma (MM) (Non Patent Literatures 3 to 7).

Recently, a phase I first-in-human (FIH) clinical study (FIH) of YS110 in solid tumors expressing CD26 (MM 23, RCC 9, urothelial carcinoma (UTC) 1) was conducted (Non Patent Literatures 8), demonstrating that YS110 therapy has a good safety profile and promotes disease control in patients with progressive/refractory tumors.

In treatment of cancer, it is very important in a therapeutic strategy to determine whether or not a therapeutic effect is achieved. In a case of a solid cancer, for example, whether or not an effect is achieved in the solid cancer is determined by confirming the effect based on RECIST or the like four weeks after administration of a therapeutic agent. However, use of biomarkers is expected as a faster and easier method of evaluating the efficacy.

Biomarkers in cancer management can be used for prophylaxis, diagnosis, and therapy selection, as well as potentially therapeutic monitoring. Markers such as EGFR or ALK fusion gene (lung cancer), HER2 (breast cancer or gastric cancer), or RAS (colon cancer) are used to select an optimal therapy by identifying selected genetic alterations. However, serum biomarkers that indicate prediction results during a cancer treatment process have not been identified so far.

Serum levels of soluble CD26 have been previously evaluated as potential biomarkers. In patients with urothelial cancer, gastric cancer, pancreatic cancer, thyroid cancer or lung cancer, a correlation between baseline serum soluble CD26 titer and clinical efficacy of treatment has been reported (Non Patent Literatures 9 to 14). It has been reported that serum soluble CD26 titer variation after a colon cancer surgery is also a predictive biomarker of risk of recurrence or metastasis (Non Patent Literatures 15 to 17). Furthermore, treatment with DPP4 inhibitor sitagliptin after a colorectal cancer or lung cancer surgery in diabetic patients was associated with longer overall survival than treatment with other antidiabetic drugs (Non Patent Literature 18), suggesting that soluble CD26/DPP4 may play some role in regulating antitumor activity. However, there are no reports that the serum soluble CD26 titer variation during a treatment process is a prognostic marker of treatment outcome.

### CITATION LIST

### Patent Literature

Patent Literature 1: WO 02/14462 A
Patent Literature 2: WO 2007/014169 A
Patent Literature 3: WO 2008/114876 A

### Non Patent Literature

Non Patent Literature 1: Ohnuma K, et al., Trends Immunol. 2008; 29(6): 295-301.
Non Patent Literature 2: Ohnuma K, et al., Front Biosci (Landmark Ed). 2018; 23:1754-79.
Non Patent Literature 3: Ho L, et al., Clin Cancer Res. 2001; 7(7): 2031-40.
Non Patent Literature 4: Inamoto T, et al., Clin Cancer Res. 2006; 12(11 Pt 1): 3470-7.
Non Patent Literature 5: Inamoto T, et al., Clin Cancer Res. 2007; 13(14): 4191-200.
Non Patent Literature 6: Yamamoto J, et al., Br J Cancer. 2014; 110(9): 2232-45.
Non Patent Literature 7: Nishida H, et al., Blood Cancer J. 2018; 8(11): 99.
Non Patent Literature 8: Angevin E, et al., Br J Cancer. 2017; 116(9): 1126-34.
Non Patent Literature 9: Liang P I, et al., Oncotarget. 2017; 8(2): 2995-3008.
Non Patent Literature 10: Boccardi V, et al., BMC Cancer. 2015; 15:703.
Non Patent Literature 11: Abooshahab R, et al., Exp Oncol. 2018; 40(4): 299-302.
Non Patent Literature 12: Sanchez-Otero N, et al., Sci Rep. 2014; 4:3999.
Non Patent Literature 13: Ye C et al., Transl Cancer Res. 2016; 5:512-519.
Non Patent Literature 14: Enz N, et al., Pharmacol Ther. 2019; 198:135-59.
Non Patent Literature 15: De Chiara L, et al., PLOS One. 2014; 9(9): e107470.
Non Patent Literature 16: De Chiara L, et al., Dis Markers. 2020; 2020:4347936.
Non Patent Literature 17: Larrinaga G, et al., PLOS One. 2015; 10(3): e0119436.
Non Patent Literature 18: Ali A, et al., Mol Clin Oncol. 2019; 10(1): 118-24.

### SUMMARY OF INVENTION

In a phase I FIH clinical study with a humanized antibody YS110 in patients with CD26-expressing tumors, a transient decrease in serum soluble CD26/DPP4 titer and subsequent recovery were observed during four weeks of a first cycle of YS110 administration. In this study, the correlation between soluble CD26/DPP4 titer variation and efficacy measures determined by response according to RECIST criteria or progression-free survival (PFS) was analyzed in a total of 26 evaluable cases or stratified groups to identify possible prognostic biomarkers for YS110 treatment.

### BRIEF DESCRIPTION OF DRAWINGS

FIG. 1 shows changes in serum soluble CD26 levels after YS110 administration by boxplot analysis. Each figure showed the serum soluble CD26 titer variation from a baseline (100% on Day 1 before administration) to before/after YS110 administration on Day 1, Day 15, and Day 29. The analyzed data were stratified into (A) a total of 26 cases, (B) 18 cases with Q2W administration, (C) 14 cases of male with Q2W administration, (D) 8 cases with Q1W administration, (E) 19 cases of MM, (F) 12 cases of MM with Q2W administration, (G) 9 cases of male and MM with Q2W administration, and (H) 6 cases of RCC. The data are shown as a mean value±a standard deviation for each group.

FIG. 2 shows a correlation between serum soluble CD26 levels and DPP4 enzyme activity. FIG. 2(A) shows the correlation between serum soluble CD26 levels (ng/ml) and serum DPP4 enzyme activity (μM/min). FIG. 2(B) shows the correlation between serum soluble CD26 titer variation from baseline (%) and serum DPP4 titer variation from baseline (%). Both were examined by a non-zero correlation.

FIG. 3 shows a relationship between serum soluble CD26 titer variation and tumor volume variation by scatter plot analysis. From the baseline of YS110 administration (100% on Day 1 before administration) for a total of 25 cases, the serum soluble CD26 titer variation on (A) Day 1 after administration, (B) Day 15 before administration, (C) Day 15 after administration, (D) Day 29 before administration, and (E) Day 29 after administration, and the tumor volume variation by RECIST response criteria on Day 43 were plotted. The data were divided into an SD (grey circles) cohort and a PD (white circles) cohort. Variation from baseline in serum soluble CD26 titer before YS110 administration on Day 29 and the tumor volume variation according to RECIST response criteria on Day 43 of (F) 17 cases with Q2W administration, (G) 14 cases of male with Q2W administration, (H) 18 cases of MM, (I) 11 cases of MM with Q2W administration, (J) 9 cases of MM and male with Q2W administration, and (K) 6 cases of RCC with Q2W administration were plotted.

FIG. 4 shows differences in the serum soluble CD26 titer variation between the SD cohort and the PD cohort by bar graph analysis. Differences between the SD cohort and the PD cohort in the serum soluble CD26 titer variation from the baseline (100% on Day 1 before administration) before YS110 administration on Day 1, Day 15, and Day 29 were analyzed. The analyzed data were stratified into (A) a total of 23 cases, (B) 17 cases with Q2W administration, (C) 14 cases of male with Q2W administration, (D) 8 cases with Q1W administration, (E) 17 cases of MM, (F) 11 cases of MM with Q2W administration, (F) 9 cases of MM and male with Q2W administration, and (H) 6 cases of RCC with Q2W administration. The data are shown as a mean value±a standard deviation for each group.

FIG. 5 shows cell surface protein expression of CD26 in human tumor cells and non-tumor cells. The indicated malignant mesothelioma cell lines (A) or non-tumor cells (B) were stained with PE-labeled mouse IgG1, kappa isotype control (BioLegend, clone MOPC-21 (i)) or PE-labeled mouse anti-human CD26 mAb (BD Biosciences, clone M-A261 (ii)). Cell surface expression of CD26 was analyzed by flow cytometry. Two-dimensional dot plots (horizontal axis: CD26, vertical axis: non-stained) (a top panel) and histograms of CD26 intensity (red lines) and isotype control gated for live cells (grey areas) (a bottom panel) are shown. Representative plots and histograms of three independent experiments were shown, and similar results were obtained in each experiment. Among the cell lines used in this experiment, CD26 was clearly expressed on cell surfaces of MSTO-CD26, JMNctrl-shRNA, H226, TIG-1, and HDMVEC. On the other hand, CD26 was hardly expressed in a parent strain of MSTO, JMN CD26-shRNA, MCF 10A, and HUVEC, and partially expressed in MeT-5A.

FIG. 6 shows that addition of YS110 reduced production of soluble CD26 from CD26 positive tumor cells and non-tumor cells. In FIGS. 6(A) and 6(B), MM cell lines (MSTO parent, MSTO-CD26, JMNctrl-shRNA, JMNCD26-shRNA or H226 cells (3.5×104 each)) (A) or non-tumor cells (MCF10A (1.0×105), HUVEC (9.0×104), MeT-5A (6.0×104), TIG-1 (5.0×104) or HDMVEC cells (9.0×104)) (B) were incubated with a control human IgG (hIgG) or humanized anti-CD26 monoclonal antibody YS110 (10 μg/ml each) for 72 hours. In FIG. 6(C), MSTO-CD26 or TIG-1 cells were incubated with a specified concentration of YS110 for three days. In FIG. 6(D), MSTO-CD26 cells were incubated with a specified concentration of YS110 for one day, three days or seven days. The concentration of soluble CD26 in a culture supernatant was measured by an ELISA method. A dashed line indicates a detection limit (0.488 ng/ml), ND indicates “undetected” (less than the detection limit). Representative data from three independent experiments are shown as mean values±standard deviations (SD) of four samples, when the values of YS110 versus vehicle or control human IgG (*p<0.01) were compared, similar results were obtained in each experiment.

### DESCRIPTION OF EMBODIMENTS

As used in the present description, “treatment” is an approach for obtaining beneficial or desired clinical results. For purposes of the present invention, the beneficial or desired clinical results include, but are not limited to, one or more of alleviation of symptoms, decrease of an extent of disease, stabilized (that is, non-deteriorated) state of disease, delay or slowing of disease progression, recovery or palliation, and (partial or total) remission of disease state, whether detectable or undetectable. “Treatment” also means extending life expectancy relative to the expected life expectancy in a case where a patient did not receive treatment.

An “effective amount” is an amount sufficient to achieve beneficial or desired clinical results, including clinical results. The effective amount can be administered in one or multiple administrations. For the purposes of the present invention, the effective amount of a pharmaceutical composition described in the present description is an amount sufficient to delay the progression of a condition associated with tumor growth. As will be understood in the art, for example, the effective amount of a pharmaceutical composition may vary or depend on other factors, such as, a patient medical history, and a type (and/or the amount) of the pharmaceutical composition to be used, among others.

The pharmaceutical composition of the present invention may contain a pharmaceutically acceptable carrier. As used in the present description, a “pharmaceutically acceptable carrier” or “pharmaceutically acceptable excipient” includes any material that, when combined with an active ingredient, is capable of maintaining biological activity of the active ingredient, is non-reactive with a subject's immune system when delivered, and is non-toxic to the subject. Examples include, but are not limited to, phosphate buffered saline, water, emulsions such as oil/water emulsions, and any standard pharmaceutical carrier such as various types of wetting agents. Preferred diluents for spraying administration or parenteral administration are phosphate buffered saline or saline (0.9%). Compositions containing such carriers are formulated by well-known conventional methods (see, e.g., Remington's Pharmaceutical Sciences, 18th edition, A. Gennaro, Ed., Mack Publishing Co., Easton, PA, 1990 and Remington, The Science and Practice of Pharmacy 20th Ed. Mack Publishing, 2000).

In certain embodiments, an “anti-CD26 antibody” as used in the present description is specifically bound to human CD26. In certain embodiments, the anti-CD26 antibody described in the present description is bound to the same epitope as YS110. In certain embodiments, the anti-CD26 antibody described in the present description is capable of blocking binding between YS110 and CD26 in a competitive assay (competing with YS110). The competition assay can be performed by bringing a test antibody into contact with an immobilized epitope or antigen, then removing an unbound antibody by washing, then bringing a labeled YS110 antibody into contact with the epitope or antigen and removing the unbound antibody by washing, and then detecting a bound YS110 antibody. Compared to the binding of the YS110 antibody to the epitope or antigen to a control that has not been contacted with the test antibody, in a case where the binding of the YS110 antibody to the epitope or antigen is reduced when the test antibody is contacted, it can be determined that there is an ability to block the binding of the YS110 antibody in the competitive assay (competing with the YS110 antibody).

Examples of binding affinity of an anti-CD26 antibody to human CD26 as used in the present description includes an affinity with a dissociation constant (that is, Kd) of less than 10⁻⁵ M, less than 5×10⁻⁵ M, less than 10⁻⁶ M, less than 5×10⁻⁷ M, less than 10⁻⁷ M, less than 5×10⁻⁸ M, less than 10⁻⁸ M, less than 5×10⁻⁹ M, less than 10⁻⁹ M, less than 5×10⁻¹⁰ M, less than 10⁻¹⁰ M, less than 5×10⁻¹¹ M, or less than 10⁻¹¹ M. The dissociation constant may also be greater than or equal to 10⁻¹⁵ M, greater than or equal to 5×10⁻¹⁵ M, greater than or equal to 10⁻¹⁴ M, greater than or equal to 5×10⁻¹⁴ M, greater than or equal to 10⁻¹³ M, greater than or equal to 5×10⁻¹³ M, greater than or equal to 10⁻¹² M, greater than or equal to 5×10⁻¹² M, greater than or equal to 10⁻¹¹ M, greater than or equal to 5×10⁻¹¹ M, greater than or equal to 10⁻¹⁰ M, or greater than or equal to 5×10⁻¹⁰ M. Methods for determining affinity are known in the art. For example, binding affinity may be determined using a BIAcore biosensor, a KinExA biosensor, a scintillation proximity assay, ELISA, ORIGEN immunoassay (IGEN), fluorescence quenching, fluorescence transfer, and/or yeast display. Affinities may also be screened using an appropriate bioassay.

One method for determining the binding affinity of an antibody to CD26 is to measure the affinity of a monofunctional Fab fragment of the antibody. To obtain the monofunctional Fab fragment, the antibody, for example, IgG, can be cleaved with papain or expressed by recombinant techniques. The affinity of an anti-CD26 Fab fragment of a monoclonal antibody can be determined by a surface plasmon resonance (SPR) system (BIAcore 3000 (trademark), BIAcore, Piscaway, NJ). An SA chip (streptavidin) is used according to a supplier's instruction manual. Biotinylated CD26 can be diluted in HBS-EP (100 mM HEPES pH 7.4, 150 mM NaCl, 3 mM EDTA, 0.005% P20) and injected onto the chip at a concentration of 0.005 mg/mL. Using variable flow time periods across individual chip channels, two ranges of antigen density are achieved: 10 to 20 response units (RU) for detailed kinetic testing and 500 to 600 RU for concentration. A mixture of Pierce elution buffer and 4 M NaCl (2:1) efficiently removes bound Fab while preserving the activity of CD26 on the chip for more than 200 injections. HBS-EP buffer can be used for all BIAcore assays as running buffer. A step-wise diluted solution of purified Fab samples (0.1 to 10× estimated KD) is injected at 100 L/min for two minutes, and a dissociation time up to 30 minutes is usually tolerated. The concentration of Fab protein can be determined by ELISA and/or SDS-PAGE electrophoresis using standard Fab of known concentrations (determined by amino acid analysis). A binding rate (kon) and a dissociation rate (koff) of a reaction rate are obtained simultaneously by fitting the data into a 1:1 Langmuir binding model (Lofas & Johnson, 1990) using the BIAevaluation program. An equilibrium dissociation constant (KD) value is calculated as koff/kon.

The compositions of the invention are useful in the treatment of conditions associated with CD26 expression (such as diseases or disorders), for example, malignant mesothelioma. In certain embodiments, the compositions of the invention may have one or more of the following characteristics: (a) binding to CD26; (b) modulating CD26 activity; (c) stopping a cell cycle of CD26+ cells at the G1/S checkpoint; (d) inhibiting growth of cells expressing CD26 (for example, malignant mesothelioma); (e) inhibiting binding of CD26 to extracellular matrix; and/or (f) being useful in the treatment of conditions associated with CD26 expression. In certain embodiments, the condition associated with expression of CD26 is a disease or disorder associated with overexpression of CD26. In certain embodiments, the condition associated with the expression of CD26 is at least partially mediated by CD26. In certain embodiments, the condition associated with the expression of CD26 is a condition associated with the growth of cells expressing CD26. In certain embodiments, the disease or disorder is cancer (for example, malignant mesothelioma, lung cancer, kidney cancer, liver cancer or other malignant tumors with the expression of CD26).

In certain embodiments, the anti-CD26 antibody includes both a heavy chain variable region containing an amino acid sequence having at least about 80% identity to the amino acid sequence selected from a group consisting of SEQ ID NOs: 8 to 14 and a light chain variable region containing an amino acid sequence having at least about 80% identity to the amino acid sequence selected from the group consisting of SEQ ID NOs: 1 to 7. In certain embodiments, the anti-CD26 antibody contained in the pharmaceutical composition of the present invention includes a light chain variable region containing an amino acid sequence having at least about 80% identity to the amino acid sequence selected from the group consisting of SEQ ID NOs: 8 to 14 and a heavy chain variable region containing an amino acid sequence having at least about 80% identity to the amino acid sequence selected from the group consisting of SEQ ID NOs: 1 to 7.

In certain embodiments, the anti-CD26 antibody includes at least 5 consecutive amino acids, at least 8 consecutive amino acids, at least about 10 consecutive amino acids, at least about 15 consecutive amino acids, at least about 20 consecutive amino acids, at least about 30 consecutive amino acids, or at least about 50 consecutive amino acids of the amino acid sequence of any one of SEQ ID NOs: 1 to 14.

The anti-CD26 antibody may be a fragment of an antibody sequence as described in the present description, and includes a fragment of at least about 50 amino acids, at least about 75 amino acids, or at least about 100 amino acids in length.

SEQ ID NO: 15
EVQLVX1SGX2X3X4X5QPGX6X7LRLX8CX9ASGX10X11LX12
TYGVHWVRQAPGKGLEWX13GVIWGX14GRTDYDX15X16FMSRV
TISX17DX18SKX19TX20YLQX21NSLRAEDTAVYYCX22RX23
RHDWFDYWGQGTTVTVSS

In the above sequence, X1 is E or Q, X2 is A or G, X3 is G or E, X4 is L or V, X5 is V, K or E, X6 is G or E, X7 is T or S, X8 is T or S, X9 is T or K, X10 is F or Y, X11 is S or T, X12 is T, N or S, X13 is V or M, X14 is G or D, X15 is A or S, X16 is A or S, X17 is K or R, X18 is N or T, X19 is S or N, X20 is V or A, X21 is M or L, X22 is M or V, M, or T, and X23 is N or S.

SEQ ID NO: 16
XIIX2X3TQSPSSLSX4X5X6GX7RX8TIX9CX10ASQX11IRN
X12LNWYQQKPGQAPRLLIYYSSNLXI3X14GVPX15RFSGSGS
GTDFTLTISRLX16X17EDX18AX19YYCQQSX20KLPX21TFG
SGTKVEIK

In the above sequence, X1 is D or E, X2 is L or E, X3 is M or L, X4 is A or V, X5 is S or T, X6 is L, P or A, X7 is D or E, X8 is V or A, X9 is T or S, X10 is S or R, X11 is G or D, X12 is S or N, X13 is H or Q, X14 is S or T, X15 is S, D or A, X16 is E or Q, X17 is P or A, X18 is F or V, X19 is T, A or I, X20 is I or N, and X21 is F or L.

Table 1 shows the amino acid sequences of humanized VL variants that are X376 (SEQ ID NO: 1), X377 (SEQ ID NO: 2), X378 (SEQ ID NO: 3), X379 (SEQ ID NO: 4), X380 (SEQ ID NO: 5), X381 (SEQ ID NO: 6), and X394 (SEQ ID NO: 7). A scheme with a Kabat number and a sequence number matches light chain variable regions.

Table 2 shows the amino acid sequences of humanized VH variants that are X384 (SEQ ID NO: 8), X385 (SEQ ID NO: 9), X386 (SEQ ID NO: 10), X387 (SEQ ID NO: 11) and X388 (SEQ ID NO: 12), X399 (SEQ ID NO: 13) and X420 (SEQ ID NO: 14). Both the scheme with a sequence number and the scheme with a Kabat number are shown. The scheme with a Kabat number includes 82a, 82b, and 82c.

TABLE 1

| | |
|---|---|
| Sequential | 10 20 30 40 50 |
| Numbering | 1234567890123456789012345678901234567890123456789012345 |
| | <--------FR1----------><---CR1---><----FR2-----><CDR2-> |
| CM03 VL | DIQMTQSPSSLSASLGDRVTITCSASQGIRNSLNWYQQKPDGAVKLLIYYSSNLH |
| X376 | DILMTQSPSSLSASPGDRVTISCRASQDIRNNLNWYQQKPGQAPRLLIYYSSNLH |
| X377 | EIELTQSPSSLSVSLGDRVTISCSASQDIRNNLNWYQQKPGQAPRLLIYYSSNLQ |
| X378 | DIEMTQSPSSLSASAGERVTISCRASQGIRNSLNWYQQKPGQAPRLLIYYSSNLQ |
| X379 | DILLTQSPSSLSATPGERATITCRASQGIRNNLNWYQQKPGQAPRLLIYYSSNLQ |
| X380 | EIEMTQSPSSLSVSAGERATISCSASQDIRNSLNWYQQKPGQAPRLLIYYSSNLH |
| X381 | EIELTQSPSSLSVSPGDRVTISCSASQDIRNSLNWYQQKPGQAPRLLIYYSSNLH |
| X394 | DILMTQSPSSLSASPGDRVTISCRASQDIRNNLNWYQQKPGQAPRLLIYYSSNLQ |
| Kabat | 24 34 50 |
| Numbering | |

(same as sequential numbering; no insertion)

| | |
|---|---|
| Sequential | 60 70 80 90 100 |
| Numbering | 6789012345678901234567890123456789012345678901234567 |
| | <--------------FR3---------------><-CDR3--><--FR4---> |
| CM03 VL | SGVPSRFSGSGSGTDFSLTISNLEPEDIATYYCQQSIKLPFTFGSGTKLEIK |
| X376 | SGVPDRFSGSGSGTDFTLTISRLEPEDFAAYYCQQSIKLPLTFGSGTKVEIK |
| X377 | TGVPARFSGSGSGTDFTLTISRLEPEDVAAYYCQQSIKLPLTFGSGTKVEIK |
| X378 | TGVPSRFSGSGSGTDFTLTISRLQAEDFATYYCQQSNKLPFTFGSGTKVEIK |
| X379 | SGVPSRFSGSGSGTDFTLTISRLQPEDVAAYYCQQSIKLPFTFGSGTKVEIK |
| X380 | TGVPARFSGSGSGTDFTLTISRLEPEDVAIYYCQQSNKLPLTFGSGTKVEIK |
| X381 | TGVPARFSGSGSGTDFTLTISRLQAEDFATYYCQQSIKLPLTFGSGTKVEIK |
| X394 | TGVPARFSGSGSGTDFTLTISRLEPEDFAAYYCQQSIKLPLTFGSGTKVEIK |
| Kabat | 56 89 97 |
| Numbering | |

(same as sequential numbering; no insertion)

TABLE 2

| | |
|---|---|
| Sequential | 10 20 30 40 50 |
| Numbering | 12345678901234567890123456789012345678901234567890123456789 |
| | <----------FR1----------><---CR1--><----FR2-----><-----CDR2 |
| CM03 VL | QVKLQKSGPGLVQPSQTLSLTCTVSGFSLTTYGVHWVRQSPGKGLEWLGVIWGGGRTDY |
| X384 | EVQLVESGAGVKQPGGTLRLTCTASGFSLTTYGVHWVRQAPGKGLEWLGVIWGDGRTDY |
| X385 | EVQLVQSGGGVKQPGETLRLTCTASGFSLTTYGVHWVRQAPGKGLEWLGVIWGDGRTDY |
| X386 | EVQLVESGAGVEQPSGTLRLTCTASGFSLTTYGVHWVRQAPGKGLEWMGVIWGDGRTDY |
| X387 | EVQLVESGAELVQPGGSLRLTCKASGFSLTNYGVHWVRQAPGKGLEWMGVINGGGRTDY |
| X388 | EVQLVQSGGGLKQPGETLRLSCTASGYSLTTYGVHWVRQAPGKGLEWMGVINGDGRTDY |
| X399 | EVQLVQSGGGLKQPGETLRLTCTASGFSLSTYGVHWVRQAPGKGLEWVGVINGDGRTDY |
| X420 | EVQLVESGGGLKQPGETLRLTCTASGFSLSTYGVHWVRQAPGKGLEWVGVINGDGRTDY |
| Kabat | 26 35 50 |
| Numbering | |

| | |
|---|---|
| Sequential | 60 70 80 90 100 110 |
| Numbering | 012345678901234567890123456789012345678901234567890123456 |
| | -----><------------FR3---------------><-CDR3-><---FR4----> |
| CM03 VL | DAAFISRLSISKDNSKSQVFFKMNSLQANDTATYYCVRNRHDWFDYWGQGTTVTVSS |
| X384 | DAAFMSRVTISKDTSKSTVYLQMNSLRAEDTAVYYCMRNRHDWFDYWGQGTTVTVSS |
| X385 | DAAFMSRVTISKDTSKSTAYLQMNSLRAEDTAVYYCMRNRHDWFDYWGQGTTVTVSS |
| X386 | DAAFMSRVTISRDTSKSTAYLQLNSLRAEDTAVYYCVRNRHDWFDYWGQGTTVTVSS |
| X387 | DASFMSRVTISKDTSKSTAYLQLNSLRAEDTAVYYCVRNRHDWFDYWGQGTTVTVSS |
| X388 | DSSFMSRVTISKDTSKSTAYLQLNSLRAEDTAVYYCVRNRHDWFDYWGQGTTVTVSS |
| X399 | DAAFMSRVTISKDTSKSTVYLQMNSLRAEDTAVYYCMRNRHDWFDYWGQGTTVTVSS |
| X420 | DAAFMSRVTISKDTSKSTVYLQMNSLRAEDTAVYYCMRNRNDWFDYWGQGTTVTVSS |
| Kabat | 65 abc3456789012345678901234567890123 |
| Numbering | 82 90 100 110 |
| | 95 102 |

The anti-CD26 antibody may further include SEQ ID NO: 17 or a fragment or variant thereof. In certain embodiments, the anti-CD26 antibody includes SEQ ID NO: 17. In certain embodiments, the anti-CD26 antibody includes SEQ ID NO: 17, excluding a signal sequence (One skilled in the art will readily appreciate that in certain embodiments, the signal sequence of an antibody is cleaved from the antibody). In certain embodiments, the anti-CD26 antibody includes a variable region of SEQ ID NO: 17. In certain embodiments, the anti-CD26 antibody includes an antibody (or a fragment thereof) having at least about 80%, at least about 85%, at least about 90%, at least about 95%, or at least about 98% identity to SEQ ID NO: 17. In certain embodiments, the anti-CD26 antibody is a fragment of SEQ ID NO: 17, and includes a fragment of at least about 10 amino acids, at least about 25 amino acids, at least about 50 amino acids, at least about 75 amino acids, or at least about 100 amino acids in length. In certain embodiments, the anti-CD26 antibody is bound to human CD26.

Heavy chain
(SEQ ID NO: 17)
MEWSWVFLFFLSVTTGVHSEVQLVESGAGVKQPGGTLRLTCTASG
FSLTTYGVHWVRQAPGKGLEWVGVIWGDGRTDYDAAFMSRVTISK
DTSKSTVYLQMNSLRAEDTAVYYCMRNRHDWFDYWGQGTTVTVSS
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSCDKTHTCPPCPAPELLGGPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALPAPIEKTISKAKGQ
PREPQVYTLPPSRDELTKNQVSLTCLVKGFYPSDIAVEWESNGQP
ENNYKTTPPVLDSDGSFFLYSKLTVDKSRWQQGNVFSCSVMHEAL
HNHYTQKSLSLSPGK

The anti-CD26 antibody further includes SEQ ID NO: 18, or a fragment or variant thereof. In certain embodiments, the anti-CD26 antibody includes SEQ ID NO: 18. In certain embodiments, the anti-CD26 antibody includes SEQ ID NO: 18, excluding the signal sequence. (One skilled in the art will readily appreciate that in certain embodiments, the signal sequence of an antibody is cleaved from the antibody.) In certain embodiments, the anti-CD26 antibody includes the variable region of SEQ ID NO: 18. In certain embodiments, the anti-CD26 antibody includes an antibody (or a fragment thereof) having at least about 80%, at least about 85%, at least about 90%, at least about 95%, or at least about 98% identity to SEQ ID NO: 18. In certain embodiments, the anti-CD26 antibody is a fragment of SEQ ID NO: 18, and includes a fragment of at least about 10 amino acids, at least about 25 amino acids, at least about 50 amino acids, at least about 75 amino acids, or at least about 100 amino acids in length. In certain embodiments, the anti-CD26 antibody further includes SEQ ID NO: 18, or a fragment or variant thereof. In certain embodiments, the anti-CD26 antibody is bound to human CD26. For example, in certain embodiments, the anti-CD26 antibody is an antibody including at least one heavy chain (for example, two heavy chains) each containing SEQ ID NO: 17 without a signal sequence and at least one light chain (for example, two light chains) each containing SEQ ID NO: 18 without a signal sequence.

Light chain
(SEQ ID NO: 18)
MSVPTQVLGLLLLWLTDARCDILLTQSPSSLSATPGERATITCRA
SQGIRNNLNWYQQKPGQAPRLLIYYSSNLQSGVPSRFSGSGSGTD
FTLTISRLQPEDVAAYYCQQSIKLPFTFGSGTKVEIKRTVAAPSV
FIFPPSDEQLKSGTASVVCLLNNFYPREAKVQWKVDNALQSGNSQ
ESVTEQDSKDSTYSLSSTLTLSKADYEKHKVYACEVTHQGLSSPV
TKSFNRGEC

In the present description, the humanized anti-CD26 antibody “YS110” means an antibody in which a heavy chain constant region consists of the amino acid sequence set forth in SEQ ID NO: 17 and a light chain constant region consists of the amino acid sequence set forth in SEQ ID NO: 18. It has been reported that YS110 is taken up into cells when it binds to CD26 on a malignant tumor cell membrane, and then transferred into nucleus (Yamada K et al, Plos One, 2013 Apr. 29; 8(4): e62304). More specifically, it is considered that YS110 is taken up into cytoplasm by Caveolin-dependent endocytosis and then transported into the nucleus by early endocytic vesicles. In other words, the anti-CD26 antibody of the present invention may be an antibody that is taken up into cells when it binds to CD26 on the malignant tumor cell membrane and then transferred into the nucleus. Whether or not the antibody is taken up into cells when it binds to CD26 on the malignant tumor cell membrane and then transferred into the nucleus can be determined by bringing the labeled antibody into contact with the cell, then detecting a position where the antibody exists based on the label using a microscope or the like, and determining a positional relationship between the position and a tissue in the cell.

In another aspect, the anti-CD26 antibody binds to one or more peptides selected from the group consisting of YSLRWISDHEYLY (SEQ ID NO: 19; Peptide 6), LEYNYVKQWRHSY (SEQ ID NO: 20; Peptide 35), TWSPVGHKLAYVW (SEQ ID NO: 21; Peptide 55), LWWSPNGTFLAYA (SEQ ID NO: 22; Peptide 84), RISLQWLRRIQNY (SEQ ID NO: 23; Peptide 132), YVKQWRHSYTASY (SEQ ID NO: 24; Peptide 37), EEEVFSAYSALWW (SEQ ID NO: 25; Peptide 79), DYSISPDGQFILL (SEQ ID NO: 26; Peptide 29), SISPDGQFILLEY (SEQ ID NO: 27; Peptide 30), and IYVKIEPNLPSYR (SEQ ID NO: 28; Peptide 63). In certain embodiments, the anti-CD26 antibody specifically binds to one or more of the peptides. These peptides are regions of human CD26. In certain embodiments, the anti-CD26 antibody, compared to one or more peptides corresponding to other regions of human CD26, specifically binds to one or more peptides selected from the group consisting of YSLRWISDHEYLY (SEQ ID NO: 19; Peptide 6), LEYNYVKQWRHSY (SEQ ID NO: 20; Peptide 35), TWSPVGHKLAYVW (SEQ ID NO: 21; Peptide 55), LWWSPNGTFLAYA (SEQ ID NO: 22; Peptide 84), RISLQWLRRIQNY (SEQ ID NO: 23; Peptide 132), YVKQWRHSYTASY (SEQ ID NO: 24; Peptide 37), EEEVFSAYSALWW (SEQ ID NO: 25; Peptide 79), DYSISPDGQFILL (SEQ ID NO: 26; Peptide 29), SISPDGQFILLEY (SEQ ID NO: 27; Peptide 30), and IYVKIEPNLPSYR (SEQ ID NO: 28; Peptide 63).

In certain embodiments, the anti-CD26 antibody binds to each of the following peptides: YSLRWISDHEYLY (SEQ ID NO: 19; Peptide 6); LEYNYVKQWRHSY (SEQ ID NO: 20; Peptide 35); TWSPVGHKLAYVW (SEQ ID NO: 21; Peptide 55); LWWSPNGTFLAYA (SEQ ID NO: 22; Peptide 84); and RISLQWLRRIQNY (SEQ ID NO: 23; Peptide 132). In certain other embodiments, the anti-CD26 antibody binds to each of the following peptides: YSLRWISDHEYLY (SEQ ID NO: 19; Peptide 6); TWSPVGHKLAYVW (SEQ ID NO: 21; Peptide 55); RISLQWLRRIQNY (SEQ ID NO: 23; Peptide 132); YVKQWRHSYTASY (SEQ ID NO: 24; Peptide 37); and EEEVFSAYSALWW (SEQ ID NO: 25; Peptide 79). In certain embodiments, the anti-CD26 antibody binds to each of the following peptides: DYSISPDGQFILL (SEQ ID NO: 26; Peptide 29); SISPDGQFILLEY (SEQ ID NO: 27; Peptide 30); and TWSPVGHKLAYVW (SEQ ID NO: 21; Peptide 55). In certain other embodiments, the anti-CD26 antibody binds to each of the following peptides:

(SEQ ID NO: 26; Peptide 29)
DYSISPDGQFILL;
(SEQ ID NO: 27; Peptide 30)
SISPDGQFILLEY;
(SEQ ID NO: 21; Peptide 55)
TWSPVGHKLAYVW;
and
(SEQ ID NO: 28; Peptide 63)
IYVKIEPNLPSYR.

Using a competitive assay, it is possible to determine whether two antibodies bind to the same epitope by recognizing the same or sterically overlapping epitope. Usually, the antigen is immobilized on a multi-well plate and an ability of the unlabeled antibody to block the binding of the labeled antibody is measured. Common labels for such competitive assays are radioactive labels or enzyme labels. Furthermore, by using epitope mapping techniques known to those skilled in the art, it is possible to determine the epitope to which the antibody binds.

In certain embodiments, the anti-CD26 antibody includes one or more constant regions. In certain embodiments, the anti-CD26 antibody includes a human constant region. In certain embodiments, the constant region is a constant region of a heavy chain. In another embodiment, the constant region is a constant region of a light chain. In certain embodiments, the anti-CD26 antibody includes a constant region having at least about 80%, at least about 85%, at least about 90%, at least about 95%, at least about 98%, or 100% identity to the human constant region. In certain embodiments, the anti-CD26 antibody includes an Fc region. In certain embodiments, the anti-CD26 antibody includes a human Fc region. In certain embodiments, the anti-CD26 antibody includes an Fc region having at least about 80%, at least about 85%, at least about 90%, at least about 95%, at least about 98%, or 100% identity to the human Fc region.

In certain embodiments, the anti-CD26 antibody is an IgG antibody. In certain embodiments, the anti-CD26 antibody is an IgG1 antibody. In another embodiment, the anti-CD26 antibody is an IgG2 antibody. In certain embodiments, the anti-CD26 antibody is a human IgG antibody.

The anti-CD26 antibody may be an antibody in a form of a monomer, a dimer, and a multimer. For example, a bispecific antibody, a monoclonal antibody having binding specificity to at least two different antigens, can be prepared using the antibody disclosed in the present description (see, e.g., Suresh et al., Methods in Enzymology, 1986, 121, 210). Conventionally, recombinant production of the bispecific antibody was based on co-expression of two sets of heavy chain-light chain pairs of immunoglobulin, including two heavy chains with different specificities (Millstein, Cuello, Nature, 1983, 305, 537-539).

According to one approach for preparing the bispecific antibody, an antibody variable region (an antibody-antigen binding site) with a desired binding specificity is fused to a constant region of the immunoglobulin. Preferably, the fusion portion involves a heavy chain constant region of the immunoglobulin that includes at least portions of a hinge part, and CH2 and CH3 regions. It is preferred that at least one fusion portion has a first heavy chain constant region (CH1) including a site essential for the binding of light chains. DNA encoding immunoglobulin heavy chain fusion and, if desired, DNA encoding immunoglobulin light chains are inserted into separate expression vectors and co-transfected to an appropriate host organism. In embodiments where an unequal ratio of three antibody chains used in construction provides an optimum yield, it is possible to prepare a mutual proportion of these three antibodies with high flexibility. In a case where expression of at least two antibodies in an equal ratio results in a high yield, or the ratio is not particularly important, coding sequences of two or all three antibodies may be inserted into one expression vector.

One approach is a bispecific antibody composed of a hybrid immunoglobulin heavy chain with a first binding specificity of one arm and a hybrid heavy chain-light chain pair (with a second binding specificity) of the other arm. This asymmetric structure (having an immunoglobulin light chain in only half of a bispecific antibody molecule) facilitates separation of a desired bispecific compound from undesired immunoglobulin chain combinations.

This approach is described in WO 94/04690 published Mar. 3, 1994.

Hetero-binding antibodies, including two covalently bound antibodies, are also within the range of anti-CD26 antibodies. Such antibodies have been used to target immune system cells to unwanted cells (U.S. Pat. No. 4,676,980) or to treat HIV infection (WO 91/00,360 and WO 92/200, 373; EP 03089). Hetero-binding antibodies may be prepare using any convenient crosslinking method. Suitable crosslinking agents and crosslinking techniques are well known in the art and are described in U.S. Pat. No. 4,676,980.

The term “anti-CD26 antibody” may include an antigen binding fragment of an anti-CD26 antibody. For example, in certain embodiments, the antibody is selected from the group consisting of Fab, Fab′, Fab′-SH, Fv, scFv, and F(ab′)₂. In certain embodiments, the antibody is a Fab. Various techniques have been developed for the production of antibody fragments. These fragments can be obtained via protein digestion of complete antibodies (see, e.g., Morimoto et al., 1992, J. Biochem. Biophys. Methods 24:107-117 and Brennan et al., 1985, Science 229:81) or can also be produced directly by recombinant host cells. For example, Fab′-SH fragments can be recovered directly from E. coli and chemically bound to form F(ab′)2 fragments (Carter et al., 1992, Bio/Technology 10:163-167). In another embodiment, F(ab′)2 is formed using a leucine zipper GCN4 that facilitates assembly of F(ab′)2 molecules. According to other approaches, fragments of Fv, Fab, or F(ab′)2 are isolated directly from recombinant host cell culture.

In certain embodiments, the anti-CD26 antibody is a single chain (ScFv), variant, fusion protein including an antibody moiety, humanized antibody, chimeric antibody, diabody, linear antibody, single-chain antibody, and immunoglobulin molecule of any other modified configuration.

Single-chain variable region fragments are prepared by linking light chain and/or heavy chain variable regions using short linking peptides. Bird et al. (1988) Science 242:423-426. An example of a linking peptide is (GGGGS)3 (SEQ ID NO: 29), which crosslinks a carboxy terminus of one variable region and an amino terminus of the other variable region by about 3.5 nm. Other sequence linkers have also been designed and used. Bird et al. (1988). The linker can then be modified for additional functions such as immobilization of a drug or immobilization to a solid carrier. Single-chain variants can be produced by either recombinant techniques or synthetic techniques. In a case where the scFv is produced by synthesis techniques, an automated synthesizer can be used. In a case where the scFv is produced by recombinant techniques, a suitable plasmid containing a polynucleotide encoding the scFv can be introduced into eukaryote such as suitable host cells, yeast cells, plant cells, insect cells or mammalian cells, or prokaryote such as E. coli. The polynucleotide encoding the scFv of interest can be prepared by routine manipulation such as ligation of the polynucleotide. The resulting scFv can be isolated using standard protein purification techniques known in the art.

Other forms of single-chain antibodies, such as diabody, are also encompassed by anti-CD26 antibodies. Diabody is a bivalent, bispecific antibody in which a VH domain and a VL domain are expressed on a single antibody chain, but it uses linkers that are too short for these two domains to pair on the same chain, so that forcibly, these domains are forced to pair with complementary domains of another chain, and two antigen binding sites are created (see, e.g., Holliger, P. et al. (1993) Proc. Natl. Acad. Sci. USA 90:6444-6448; Poljak, R. J., et al. (1994) Structure 2:1121-1123).

While the anti-CD26 antibody encompass modifications to the antibody described in the present description, examples of such modifications include functionally equivalent antibodies and variants with enhanced or diminished activity that do not significantly affect properties of the antibody. Modifications of the antibody are routine manipulations in the art and need not be described in detail in the present description. Examples of modified antibodies include antibodies that involve conservative substitution of amino acid residues, deletion or addition of one or more amino acids that do not significantly impair functional activity, or the use of chemical analogs.

Insertion or addition of an amino acid sequence includes fusion at the amino terminus and/or carboxyl terminus from one residue to an antibody containing 100 or more residues in length and insertion within the sequence of a single or multiple amino acid residues. Examples of terminal insertion include an antibody with N-terminal methionyl residues or an antibody fused to epitope tags. Other insertion variants of an antibody molecule include fusion of an enzyme or an antibody that extends a serum half-life of the antibody to N-terminus or C-terminus of the antibody.

Substantial modifications in biological properties of the antibody are achieved by selecting a substitution that significantly changes an effect of the modification on (a) the structure of an antibody main chain in a substituted region, e.g., a sheet or helix conformation, (b) charge or hydrophobicity of the molecule at a target site, or (c) maintenance of a volume of a side chain. Residues present in nature are classified into the following groups based on common side chain properties:
(1) Hydrophobicity: norleucine, Met, Ala, Val, Leu, Ile;
(2) Neutral hydrophilicity: Cys, Ser, Thr;
(3) Acidity: Asp, Glu;
(4) Basicity: Asn, Gln, His, Lys, Arg;
(5) Residues affecting chain orientation: Gly, Pro; and
(6) Aromatic: Trp, Tyr, Phe.

Substitution of any cysteine residue that is not involved in maintaining a proper conformation of the antibody (usually substitution with serine) can improve oxidative stability of the molecule and prevent abnormal crosslinking. Conversely, especially in a case where the antibody is an antibody fragment, such as an Fv fragment, stability of the antibody can be improved by adding a cysteine bond to the antibody.

Amino acid modifications range from a change or modification of one or more amino acids to a complete redesign of a region such as a variable region. Changes in the variable region can alter binding affinity and/or specificity. In certain embodiments, less than or equal to 1 to 5 conservative amino acid substitutions are made within a CDR domain. In other embodiments, less than or equal to 1 to 3 conservative amino acid substitutions are made within a CDR3 domain. In yet another embodiment, the CDR domain is CDRH3 and/or CDR L3.

The monoclonal antibody may be prepared using a hybridoma method as described by Kohler and Milstein, 1975, Nature 256:495. In the hybridoma method, mice, hamsters, or other suitable host animals are usually immunized with an immunizing agent to induce lymphocytes that produce or are capable of producing an antibody that specifically binds to the immunizing agent. Alternatively, lymphocytes may be immunized in vitro.

The monoclonal antibody (and other antibodies) may also be prepared by a recombinant DNA method as described in U.S. Pat. No. 4,816,567. DNA encoding the monoclonal antibody is isolated and sequenced using conventional methods, such as the use of an oligonucleotide probe that can specifically bind to genes encoding heavy chains or light chains of the monoclonal antibody. After isolation, the DNA is incorporated into an expression vector and transfected into host cells such as E. coli cells, simian COS cells, Chinese hamster ovary cells (CHO), or myeloma cells that does not produce immunoglobulin protein unless the expression vector is introduced, thereby achieving synthesis of the monoclonal antibody in the recombinant host cells.

Various protein expression systems, vectors, and cell culture medium useful in the production of an antibody are known to those skilled in the art. See, for example, WO 03/054172, WO 04/009823, and WO 03/064630 (the full text of these is incorporated in the present description by reference). In certain embodiments, a glutamine synthase (GS) expression system is used for expression of the anti-CD26 antibody.

The anti-CD26 antibody is preferably a humanized antibody. A therapeutic antibody often induces side effects, partly due to inducing an immune response to the administered antibody. As a result, a decrease in drug efficacy, a decrease in the number of cells with target antigen, and an undesirable inflammatory reaction can occur. In order to avoid the above, a recombinant anti-CD26 humanized antibody may be prepared. A general principle of humanization of an antibody involves maintaining a basic sequence of an antigen binding portion of the antibody, while exchanging at least a portion of a non-human residue of the antibody for human antibody sequences. The four conventional general steps for humanizing a monoclonal antibody include, but are not limited to: (1) determining a nucleotide sequence and a putative amino acid sequence of a light chain variable domain and a heavy chain variable domain of a starting antibody; (2) determining which antibody framework region or residue and/or CDR residue is used in a step of designing, that is, humanizing a humanized antibody; (3) actual humanizing methodology/techniques; and (4) transfection and expression of the humanized antibody. In a case where the antibody is used in a clinical study and treatment in humans, a constant region can also be brought closer to a human constant region by recombinant techniques to avoid an immune response. See, e.g., U.S. Pat. Nos. 5,997,867 and 5,866,692.

In a recombinant humanized antibody, interaction with an Fcγ receptor and a complement immune system can be avoided by modifying an Fcγ moiety. Techniques for preparing such an antibody are described in WO 99/58572.

Many “humanized” antibody molecules have been reported including antigen binding sites derived from non-human immunoglobulins, examples of which include rodent V regions and their associated complementarity determining regions (CDR) fused to a human constant domain. See, e.g., Winter et al., Nature 349:293-299 (1991); Lobuglio et al., Proc. Nat. Acad. ScI USA 86:4220-4224 (1989); Shaw et al., J Immunol. 138:4534-4538 (1987); and Brown et al., Cancer Res. 47:3577-3583 (1987). Other references describe a rodent CDR incorporated into a human support framework region (FR) prior to fusion with a suitable human antibody constant domain. See, e.g., Riechmann et al., Nature 332:323-327 (1988); Verhoeyen et al., Science 239:1534-1536 (1988); and Jones et al., Nature 321:522-525 (1986). Another reference describes a rodent CDR supported in a recombinant veneered rodent framework region. See, e.g., EP 519, 596. These types of “humanized” molecules are designed to minimize unwanted immunological responses to rodent anti-human antibody molecules that limit duration and efficacy of therapeutic applications of those moieties in human transplant patients. Other available methods of humanizing an antibody are disclosed in Daugherty et al., Nucl. Acids Res., 19:2471-2476 (1991) and U.S. Pat. Nos. 6,180,377; 6,054,297; 5,997,867; 5,866,692; 6,210,671; 6,350,861; and WO 01/27160.

Further exemplary methods of humanizing an antibody are described in WO 02/084277 and US 2004/0,133,357, and the full texts of both are incorporated in the present description by reference.

Furthermore, the anti-CD26 antibody may be bound to a water-soluble polymer moiety. The anti-CD26 antibody may be bound to polyethylene glycol (PEG), monomethoxy-PEG, an ethylene glycol/propylene glycol copolymer, carboxymethyl cellulose, dextran, polyvinyl alcohol, or the like. The anti-CD26 antibody may be modified at random locations on the molecule or predetermined locations on the molecule and may include 1, 2, or 3 or more binding portions. The polymer may be of any molecular weight and may be branched or unbranched. In certain embodiments, the portion is bound to an antibody via a linker. In certain embodiments, the binding portion increases a circulating half-life of the antibody in an animal body. Methods of binding polymers such as PEG to an antibody are well known in the art. In certain embodiments, the anti-CD26 antibody is a PEGylated antibody, such as a PEGylated antibody. Further, the anti-CD26 antibody may further be bound to other drugs such as a different chemotherapeutic agent, a radionuclide, an immunotherapeutic agent, a cytokine, a chemokine, a contrast agent, a toxin, a biological agent, an enzyme inhibitor, or an antibody.

The pharmaceutical composition in the present description is a pharmaceutical composition for treating malignant mesothelioma that contains the anti-CD26 antibody described above as an active ingredient. The pharmaceutical composition of the present invention is a pharmaceutical composition for treating cancer that contains the anti-CD26 antibody as an active ingredient, the pharmaceutical composition is for administration to a patient in whom the level of soluble CD26 in the serum of a patient with malignant mesothelioma on Day 1 to Day 60 from a reference administration is less than 85% of the level of soluble CD26 in the serum of the patient before the reference administration, with a reference administration date of the anti-CD26 antibody as Day 1. The pharmaceutical composition according to claim 29 that is administrated to a patient in which the level of soluble CD26 in the serum of the patient from Day 1 to Day 30 from a reference administration of the anti-CD26 antibody is less than 60% of the level of soluble CD26 in the serum of the patient before the reference administration. The pharmaceutical composition of the present invention can be a pharmaceutical composition that is for administration to a patient in whom the level of soluble CD26 in the serum of the patient on Day 22 to Day 30 from a reference administration of the anti-CD26 antibody is less than 60% of the level of soluble CD26 in the serum of the patient before the reference administration; a pharmaceutical composition that is for administration to a patient in whom the level of soluble CD26 in the serum of the patient on Day 2 to Day 8 from a reference administration of the anti-CD26 antibody is less than 50% of the level of soluble CD26 in the serum of the patient before the reference administration; a pharmaceutical composition that is administrated once every two weeks, and is for administration to a patient in whom the level of soluble CD26 in the serum of the patient from Day 22 to Day 30 from a reference administration is less than or equal to 65% of the level of soluble CD26 in the serum of the patient before the reference administration; a pharmaceutical composition that is administrated once every two weeks, and is for administration to a patient in whom the level of soluble CD26 in the serum of the patient on Day 29 from a reference administration is less than or equal to 62.3% of the level of soluble CD26 in the serum of the patient before the reference administration; a pharmaceutical compositions that is administrated to male; a pharmaceutical composition that is administrated once a week, and is for administration to a patient in whom the level of soluble CD26 in the serum of the patient from Day 2 to Day 8 from a reference administration is less than or equal to 50% of the level of soluble CD26 in the serum of the patient before the reference administration; a pharmaceutical composition that is for administration to a patient in whom the level of soluble CD26 in the serum of the patient from Day 2 to Day 8 from a reference administration is less than or equal to 49% of the level of soluble CD26 in the serum of the patient before the reference administration; a pharmaceutical composition that is for administration to a patient in whom the level of soluble CD26 in the serum of the patient on and after Day 15 from a reference administration is less than or equal to 30% of the level of soluble CD26 in the serum of the patient before the reference administration; a pharmaceutical composition that is for administration to a patient in whom the level of soluble CD26 in the serum of the patient on and after Day 15 from a reference administration is less than or equal to 26% of the level of soluble CD26 in the serum of the patient before the reference administration; a pharmaceutical composition that is administrated at 6 mg/kg as an anti-CD26 antibody amount; a pharmaceutical composition in which the reference administration is the first administration; a pharmaceutical composition that is administrated at 0.1 to 6 mg/kg as an anti-CD26 antibody amount; a pharmaceutical composition that is administrated once every two weeks or once a week; and a pharmaceutical composition in which the anti-CD26 antibody is YS110.

In the present description, “cancer” may be, for example, malignant mesothelioma, lung cancer, kidney cancer, liver cancer, or other malignant tumor with expression of CD26.

The methods described in the present description (including therapeutic methods) may be performed by a single direct injection at a single time point or multiple time points to a single site or multiple sites. Administration may also be performed to multiple sites at almost the same time. Administration Frequency is determined and adjusted over a treatment process and is based on results desired to be achieved. In some cases, the pharmaceutical composition of the present invention and a sustained-release formulation of the pharmaceutical composition may be suitable. Various formulations and devices for achieving sustained release are well known in the art.

A subject to be treated or prevented is a human.

The pharmaceutical composition is preferably administered to a mammal in a state of being contained in a carrier (preferably a pharmaceutically acceptable carrier). Suitable carriers and their formulations are described in Remington's Pharmaceutical Sciences, 18th ed., A. Gennaro, ed., Mack Publishing Co., Easton, PA, 1990 and Remington, The Science and Practice of Pharmacy, 20th ed., Mack Publishing, 2000. Usually, use of an appropriate amount of a pharmaceutically acceptable salt in a formulation will make the formulation isotonic. Examples of carriers include saline, Ringer's solution, and dextrose solution. pH of these solutions is preferably from about 5 to about 8, more preferably from about 7 to about 7.5. Furthermore, carriers include a sustained-release formulation such as a semipermeable matrix made of a solid hydrophobic polymer containing an antibody, the matrix of which is in the form of a shaped article such as, for example, a film, liposomes or microparticles. For example, it will be apparent to those skilled in the art that certain heterologous carriers may be more preferred, depending on a route and concentration of administration of the antibody to be administered.

The pharmaceutical composition may be administered to a mammal by injection (for example, systemically, intravenously, intraperitoneally, subcutaneously, intramuscularly, intraportally) or by other methods that ensure delivery into bloodstreams in an effective form (for example, injection). The pharmaceutical composition may also be administered in an isolated perfusion method such as tissue isolated perfusion to obtain a therapeutic effect locally. Intravenous injection is preferred.

An effective dose and a schedule for administering the pharmaceutical composition of the present invention are empirically determined, and such determination methods are within a scope of technical common sense of the art. For example, once a week can be administered five times, or once every two weeks can be administered three times. Those skilled in the art will appreciate that the dose of the pharmaceutical composition to be administered will vary depending on, for example, the mammal inoculating the pharmaceutical composition, the route of administration, the specific type of the antibody to be used, and other drugs to be administered to the mammal. A typical dosage per day of a pharmaceutical composition used alone may range from about 1 μg/kg body weight to 100 mg/kg body weight or more as an active ingredient amount per day, depending on the aforementioned factors. In general, any of the following dosages may be used: at least about 50 mg/kg body weight; at least about 10 mg/kg body weight; at least about 3 mg/kg body weight; at least about 1 mg/kg body weight; at least about 750 μg/kg body weight; at least about 500 μg/kg body weight; at least about 250 μg/kg body weight; at least about 100 μg/kg body weight; at least about 50 μg/kg body weight; at least about 10 μg/kg body weight; at least about 1 μg/kg body weight, or greater, is administered. Preferably, the amount of the anti-CD26 antibody is 0.1 to 2 mg/kg once every two weeks for three times or 2 to 6 mg/kg once a week for five times.

The method of the present invention is based on a higher antitumor effect in a patient in which the level of soluble CD26 in the serum is increased by administration of the anti-CD26 antibody. In other words, the method of the present invention utilizes the fact that the efficacy of the anti-CD26 antibody can be predicted by comparing the level of soluble CD26 in the serum of a cancer patient before administration of the anti-CD26 antibody with the level of soluble CD26 in the serum of the patient on and after one day from the administration. In other words, the method of the present invention requires at least one administration of the anti-CD26 antibody to a subject. Such administration of the anti-CD26 antibody that serves as a reference for comparing the level of soluble CD26 in the serum before and after a start of administration is referred to as reference administration. The reference administration is preferably an administration for the first time in the patient, but is not necessarily have to be the first administration received in the patient. For example, in a case where a patient who has received sugar of the anti-CD26 antibody in the past is administered the anti-CD26 antibody again after a lapse of an arbitrary period after receiving the latest administration, the administration may be used as the reference administration. The level of soluble CD26 in the serum of a cancer patient before administration of the anti-CD26 antibody serving as a reference for comparison (100%) is measured before the reference administration. In the method of the present invention, the level of soluble CD26 in the serum of the patient measured before the anti-CD26 antibody administration to be a reference (a reference level) is referred to as “the level of soluble CD26 in the serum of the patient before the reference administration”. The level of soluble CD26 in the serum of the patient on or after Day 1 from the reference administration, which is to be compared with the reference level (100%), is referred to as “the level of soluble CD26 in the serum of the patient on a measurement date”. Here, on and after Day 1 from the reference administration is the number of days calculated with the reference administration as Day 1. The day on which the level of soluble CD26 in the serum of the patient to be compared is measured is referred to as a measurement date. The administration of the anti-CD26 antibody received by the measurement date may be only one time of the reference administration or multiple times. For example, in a case where the anti-CD26 antibody is administered once a week, the anti-CD26 antibody is administered on Day 8, Day 15, Day 22, Day 29. In a case where the measurement date is Day 1 to Day 8 (before the administration), only the reference administration is performed, but on and after Day 8 (after the administration), two or more administrations are received. In a case where the measurement date is an administration date, the measurement of the level of soluble CD26 in the serum of the patient can be before the administration or after the administration. Further, the anti-CD26 antibody is administered as a pharmaceutical composition containing an anti-CD26 antibody as an active ingredient according to the administration of the pharmaceutical composition described above.

Therefore, in one aspect, the present invention relates to a method for selecting a cancer patient that have a potential to obtain a therapeutic effect with an anti-CD26 antibody, the method including: comparing the level of soluble CD26 in the serum of the patient before a reference administration of the anti-CD26 antibody with the level of soluble CD26 in the serum of the patient on a measurement date; and in a case where the level of soluble CD26 in the serum of the patient on the measurement date is less than 85% of the level of soluble CD26 in the serum of the patient before the reference administration, selecting the patient as having the potential to obtain a therapeutic effect with the anti-CD26 antibody, wherein the measurement date is Day 1 to Day 60 with a reference administration date as Day 1, and the patient is a patient to whom the anti-CD26 antibody is administered as a reference administration at least once; and, in a case where the measurement date corresponds to an administration date of the anti-CD26 antibody, the level of soluble CD26 in the serum of the patient on the measurement date is the level of soluble CD26 in the serum of the patient before the administration of the anti-CD26 antibody on the measurement date.

In the present description, the “reference administration” means an administration for determining whether or not the level of soluble CD26 in the serum of the patient after the administration has decreased with reference to the level of soluble CD26 in the serum of the patient before the administration, and does not need to be the first administration for the patient. However, since the efficacy of a drug is usually performed at an early stage of the start of treatment, the reference administration is preferably the first administration.

Throughout the present description, the “level” means an index related to a quantified abundance, and includes, for example, concentration, amount, or an index that can be used as a substitute thereof. Therefore, the level may be a measurement value itself such as fluorescence intensity or a value converted into concentration or the like. Further, the level may be an absolute numerical value (abundance, abundance per unit area, etc.), or may be a relative numerical value (ratio (%), multiple, etc.) compared with a comparison control set as necessary.

In another aspect, the present invention relates to a method for predicting a therapeutic effect with the anti-CD26 antibody in a cancer patient, the method including: comparing the level of soluble CD26 in the serum of the patient before a reference administration of the anti-CD26 antibody with the level of soluble CD26 in the serum of the patient on a measurement date; and in a case where the level of soluble CD26 in the serum of the patient on the measurement date is less than 85% of the level of soluble CD26 in the serum of the patient before the reference administration, predicting that the anti-CD26 antibody may have a therapeutic effect in the patient, wherein the measurement date is Day 1 to Day 60 from the reference administration with the reference administration date as Day 1, and the patient is a patient to whom the anti-CD26 antibody is administered as the reference administration at least once; and, in a case where the measurement date corresponds to the administration date of the anti-CD26 antibody, the level of soluble CD26 in the serum of the patient on the measurement date is the level of soluble CD26 in the serum of the patient before the administration of the anti-CD26 antibody on the measurement date.

In the above method, the “measurement date” can be set as Day 1 to Day 60 with the reference administration date as Day 1, and can be, for example, Day 1 to Day 30, Day 2 to Day 8, Day 2 to Day 15, Day 2 to Day 21, Day 2 to Day 29, Day 8 to Day 15, Day 8 to Day 21, Day 8 to Day 29, Day 15 to Day 22, Day 15 to Day 29, or Day 22 to Day 29, or Day 1, 2, 3, 4, 5, 6, 7, 8, 9, 10, 11, 12, 13, 14, 15, 16, 17, 18, 19, 20, 21, 22, 23, 24, 25, 26, 27, 28, or 29, or a date included in a period between any two points thereof.

In the method of the present invention, the case where it is determined that a therapeutic effect may be obtained with the anti-CD26 antibody or that the anti-CD26 antibody may have a therapeutic effect in the patient can be a case where the level of soluble CD26 in the serum of the patient on the measurement date is less than 85%, 80%, 75%, 70%, 65%, 62.3%, 60%, 55%, 50%, 45%, 40%, 35%, 30%, 26%, or 25% of the level of soluble CD26 in the serum of the patient before the reference administration.

Preferably, in a case where 6 mg/kg of the anti-CD26 antibody is administered at a frequency of once a week, when the measurement date is Day 2 to Day 8 from the reference administration, and the level of soluble CD26 in the serum of the patient on the measurement date is less than 50% (or less than 49%) of the level of soluble CD26 in the serum of the patient before the reference administration, it is determined that a therapeutic effect may be obtained with the anti-CD26 antibody, or that the anti-CD26 antibody may have a therapeutic effect in the patient.

Alternatively, preferably, in a case where 6 mg/kg of the anti-CD26 antibody is administered at a frequency of once a week, when the measurement date is on and after Day 15 (for example, Day 15 to Day 22, Day 15 to Day 29, or Day 22 to Day 29) from the reference administration and the level of soluble CD26 in the serum of the patient on the measurement date is less than 30% (or less than 26%) of the level of soluble CD26 in the serum of the patient before the reference administration, it is determined that a therapeutic effect may be obtained with the anti-CD26 antibody, or that the anti-CD26 antibody may have a therapeutic effect in the patient.

The method described above may further include any step selected from administering the anti-CD26 antibody to a patient, collecting the serum of the patient before the reference administration, and collecting the serum of the patient after the period specified above from the reference administration, and measuring soluble CD26 in the serum of the patient before the reference administration, and measuring soluble CD26 in the serum of the patient after the reference administration. The soluble CD26 can be measured using a commercially available kit such as an ELISA kit or by a method well known to those skilled in the art.

Further, in one aspect, the present invention also relates to a method for measuring the level of soluble CD26 in the serum of a cancer patient before administration of the anti-CD26 antibody and the level of soluble CD26 in the serum of the patient on Day 1 to Day 60 (preferably on Day 5 to Day 45, Day 15 to Day 30, Day 25 to Day 30) from the administration.

Further, in the methods described in the present description, the level of soluble CD26 (or measurement thereof) may be replaced with DPPIV activity (or measurement thereof). The DPPIV activity can be measured by using a commercially available kit.

Furthermore, the method of the present invention may include administering a pharmaceutical composition for treating malignant mesothelioma that contains the anti-CD26 antibody as an active ingredient to a patient selected as having a possibility of obtaining a therapeutic effect with the anti-CD26 antibody by the method or a patient predicted as having a therapeutic effect with the anti-CD26 antibody.

Hereinafter, examples will be described in order to describe the present invention in more detail, but the present invention is not limited thereto. Note that, all documents cited throughout the present application are incorporated into the present application as they are by reference.

### (1) Human Subject

In an FIH Phase I clinical study, 33 patients (MM 23, RCC 9, UTC 1) administered with YS110 were subjected to safety analysis, with 26 of 33 (MM 19, RCC 6, UTC 1) evaluated for therapeutic efficacy (Angevin E, et al., Br J Cancer. 2017; 116(9): 1126-34). To determine a maximum tolerated dose, patients first received YS110 infusions at 0.1, 0.4, 1, and 2 mg/kg for a total of three times on Day 1, Day 15, and Day 29 (once every two weeks, Q2W). Based on preliminary pharmacokinetic data, a protocol was subsequently revised to allow patients to receive YS110 infusions at 2, 4, and 6 mg/kg for a total of five times on Day 1, Day 8, Day 15, Day 22, and Day 29 (once a week, Q1W). Of the 33 patients, 26 patients (18 cases in Q2W cohort, 8 cases in Q1W cohort) were evaluable for YS110-mediated antitumor activity by RECIST criteria or PFS monitoring. The tumor volume variation from baseline was evaluated on Day 43±4.2, two weeks after completion of a first cycle of YS110 administration on Day 29, according to the modified RECIST criteria for MM, or RECIST 1.0 criteria for RCC or UTC (Angevin E, et al. (2017) supra). Serum soluble CD26/DPP4 titer were measured just before and immediately after YS110 administration on Day 1, Day 15 and Day 29.

### (2) Statistical Analysis

Boxplot analysis was used to observe serum soluble CD26/DPP4 titer variation before/after YS110 infusion on Day 1, Day 15 and Day 29. Using scatter plot analysis stratified for stable disease (SD) and progressive disease (PD) cases, a relationship between the serum soluble CD26 titer variation before/after YS110 administration on Day 1, Day 15, and Day 29 and the tumor volume variation from baseline on Day 43 was observed. For these two observational analyses, PPMC analysis or SRDC analysis was used to statistically examine a potential correlation between the serum soluble CD26 titer variation before/after YS110 administration on Day 1 and Day 29 from baseline and the tumor volume variation by RECIST criteria on Day 43. Based on Pearson's product-moment correlation/Spearman's rank-difference correlation (PPMC/SRDC) analysis, bar graph analysis of the serum soluble CD26/DPP4 titer variation stratified by SD cases and PD cases before YS110 administration on Day 1 (baseline, 100%), before YS110 administration on Day 15 and before YS110 administration on Day 29 was performed to examine the correlation between the serum soluble CD26/DPP4 variation in Wilcoxon's rank-sum test and incidence of SD cases or PD cases according to RECIST criteria on Day 43. Based on the results of PPMC/SRDC and bar graph analysis, an index of serum soluble CD26 titer variation from baseline (a cutoff titer) was examined for SD results according to RECIST criteria and PFS≥90 days or ≥180 days by Fisher's exact test using ROC analysis. Differences in background factors between SD cases and PD cases were examined by Fisher's exact test or Wilcoxon's rank-sum test prior to ROC analysis.

### (3) Cell Lines and Cultures

Human MM cell lines MSTO-211H (MSTO parent) and NCI-H226 were obtained from the American Type Culture Collection (ATCC, Rockville, Maryland). MSTO parental cells were stably transfected with full-length human CD26 (MSTO-CD26) (Yamamoto J, et al., Br J Cancer. 2014; 110(9): 2232-45). Human MM cell line JMN cells were transduced with short hairpin RNA (shRNA)-expressing lentivirus to generate stable cell lines JMNCD26-shRNA and JMNctrl-shRNA (Yamazaki H, et al., Biochem Biophys Res Commun. 2012; 419(3): 529-36). As non-tumor human cells, an immortalized pleural mesothelial cell line MeT-5A, a breast epithelial cell line MCF 10A, a fetal pulmonary fibroblast cell line TIG-1, human umbilical vein endothelial cells (HUVEC), and human skin microvascular endothelial cells (HDMVEC) were used. MeT-5A and MCF10A were obtained from ATCC and TIG-1 was obtained from JCRB Cell Bank (Japan, Osaka). Medium for HUVEC, HDMVEC, and MCF10A, HUVEC, HDMVEC (MEGM, EGM-2, EGM-2 MV, respectively) were purchased from LONZA (Walkersville, Maryland). MSTO parental, MSTO-CD26, JMNctrl-shRNA, JMNCD26-shRNA, H226, and MeT-5A were grown in RPMI1640 medium supplemented with 10% FBS. TIG-1 was grown in DMEM medium supplemented with 10% FBS. All cells were cultured in a humidified 5% CO₂ incubator at 37° C.

### (4) Antibodies and Reagents

Humanized anti-CD26 monoclonal antibody YS110 was provided by Y's AC Co., Ltd. (Tokyo, Japan). A human IgG1 isotype control monoclonal antibody (clone QA16A12) purchased from BioLegend (San Diego, California) was used as a control.

### (5) Preparation of Culture Supernatant

Cells were cultured at 37° C. for 3 days in 500 μl of medium in 24 well plates (Corning), in presence or absence of control human IgG or YS110. For time course analysis, MSTO-CD26 (1.5×10⁵, 4×10⁴, or 4×10³) was cultured in 500 μl of RPMI1640 medium in 24 well plates, in the presence or absence of YS110 (1, 3, 10 μg/mL) at 37° C. for 1, 3, or 7 days, respectively. After incubation, supernatant was collected from confluent cultures.

### (6) Quantification of Soluble CD26 and DPP4 Enzyme Activity

Assays for soluble CD26 and DPP4 activity were developed in our laboratory using a mouse anti-human CD26 monoclonal antibody (clones 5F8 and 9C11) that does not show cross-reactivity with therapeutic humanized anti-CD26 monoclonal antibody YS110. Related experimental methods have been detailed previously (Ohnuma K, et al., J Clin Lab Anal. 2015; 29(2): 106-11). Data were analyzed by Tukey's one-way ANOVA test for multiple controlled studies. Significance was analyzed using GraphPad Prism 6 (GraphPad Software, San Diego, California). Values of p<0.01 are considered significant and are shown in the corresponding figures and the description of the figures.

### (7) Changes in Serum Soluble CD26/DPP4 Titer Before/after YS110 Administration (Shown in Boxplot)

This Phase I study includes several important parameters such as: 1) Tumor histological type: MM 19 cases, RCC 6 cases, UTC 1 case; 2) YS110 dosage: 0.1 to 6 mg/kg; 3) Administration Frequency: A study of background factors between SD cases and PD cases, with 3 administrations once every two weeks (Q2W) in 18 cases and 5 administrations once a week (Q1W) in 8 cases, showed no bias in age, BMI, absolute value of tumor volume, or serum soluble CD26/DPP4 titer before YS110 administration, except for sex (data not shown). As shown in Tables 3 and 4, YS110 appeared more effective in female patients (SD 6 cases and PD 2 cases in MM, SD 1 case in RCC, PD 1 case in UTC) in contrast to male patients (SD 4 cases and PD 7 cases in MM, SD 2 cases and PD 3 cases in RCC).

TABLE 3
Demographic chart of 26 evaluable cases on administration frequency and dosage, gender,
PFS, age, BMI, RECIST evaluation, tumor volume change and serum sCD26/DPP4 titer change.

| | | | | | | | | | J | | | | K | |
|---|---|---|---|---|---|---|---|---|---|---|---|---|---|---|
| A | | | D | | | | | | Tumor | | | | sCD26 | |
| Adminis- | | C | Type | | F | G | H | | Volume (mm) | | | | Titer (ng/ml) | |
| tration | B | Patient | of | E | Dose | PFS | Age | I | Baseline | | | Ch [symbol] e | Day 1 | |
| Frequency | RECIST | NO. | Tumor | Gender | ( [symbol] ) | (Days) | (Year) | BMI | (Day 1Pre) | RECIST | | (%) | Pre | Post |
| Once | PD | 1 | MM | M | 0.1 | 41 | [symbol] 0 | 2 [symbol] | 122 | 140 | (Day 42) | 14.8 | 1007 [symbol] 2 | [symbol] 81.2 |
| per | | 2 | MM | M | 0.1 | 42 | 72 | 31 | 212 | 253 | (Day 43) | 19.3 | 1313.6 | 436.5 |
| two | | 3 | MM | F | 0.4 | 40 | 57 | 25 | N.A. | N.A. | | N.A. | 1529.4 | 494.8 |
| weeks | | 4 | MM | M | 1.0 | 40 | 64 | 24 | 15 | 16 | (Day 4 [symbol] ) | 6.7 | 661.5 | 210.0 |
| (Q2W) | | 5 | RCC | M | 1.0 | 43 | 41 | 25 | 183 | 222 | (Day 44) | 21.3 | 997.8 | 410.4 |
| | | 6 | MM | M | 1.0 | 40 | 50 | 23 | 173 | 20 [symbol] | (Day 41) | 20.2 | 87 [symbol] .9 | 318.4 |
| | | 7 | RCC | M | 1.0 | 59 | 58 | 24 | 40 | 60 | (Day [symbol] 0) | 50 [symbol] 0 | 458 [symbol] 0 | 188 [symbol] 4 |
| | | 8 | RCC | M | 2.0 | 41 | 5 [symbol] | 27 | 9 [symbol] | 109 | (Day 42) | [symbol] .2 | [symbol] 60.1 | 3 [symbol] 5.0 |
| | | 9 | MM | M | 2.0 | 39 | 56 | 25 | 29 | 37 | (Day 43) | 27.6 | 832.1 | 368.3 |
| | | 10 | MM | M | 2.0 | 42 | [symbol] 7 | 26 | 37 [symbol] | 475 | (Day 43) | 26.3 | 715.4 | 325.7 |
| | SD | 11 | MM | F | 0.1 | 42 | [symbol] 1 | 20 | 115 | 128 | (Day 43) | 11.3 | 1227. [symbol] | 406.4 |
| | | 12 | MM | M | 0.4 | 223 | 63 | 2 [symbol] 3 | 7 [symbol] | 74 | (Day 42) | −2. [symbol] | 108 [symbol] 2 | 327.9 |
| | | 13 | RCC | M | 0.4 | 273 | 50 | 24 | [symbol] 7 | 99 | (Day 43) | 2.1 | 730.7 | 222.1 |
| | | 14 | RCC | M | 2.0 | 57 | 63 | 39 | 209 | 204 | (Day 44) | −2.4 | 396.8 | 18 [symbol] .5 |
| | | 15 | RCC | F | 2.0 | 22 [symbol] | 63 | 40 | 7 [symbol] | 74 | (Day 45) | −1.3 | 6 [symbol] 5.0 | 330.4 |
| | | 16 | MM | F | 2.0 | 399 | 73 | 2 [symbol] 7 | 1 [symbol] | 139 | (Day 46) | −0.5 | 1246.4 | 585.0 |
| | | 17 | MM | M | 2.0 | 98 | [symbol] 5 | 32 | 25 | 26 | (Day 43) | 4.0 | 1139 3 | 596.2 |
| | | 18 | MM | M | 2.0 | 230 | 72 | 24 | 90 | 90 | (Day 43) | 0.0 | 1281.9 | 499.3 |
| Once | PD | 19 | UTC | F | 2.0 | 47 | [symbol] 9 | 23 | 153 | 238 | (Day 49) | 55.6 | 561.4 | 1 [symbol] 8.9 |
| a | | 20 | MM | M | 4.0 | 35 | 73 | 25 | 119 | 129.4 | (Day 43) | 8.5 | 830.8 | 385.4 |
| week | | 21 | MM | F | [symbol] .0 | 33 | 69 | 3 [symbol] | 230 | 234.4 | (Day 36) | 2.0 | 721.1 | 352.5 |
| (Q1W) | SD | 22 | MM | F | [symbol] .0 | 41 | 59 | 28 | [symbol] | 226 | (Day 43) | [symbol] .1 | 1348.4 | 758.3 |
| | | 23 | MM | F | 2.0 | 184 | 6 [symbol] | 17 | 43 | 44 | (Day 36) | 2. [symbol] | 759.2 | 230.9 |
| | | 24 | MM | M | 4.0 | 59 | 76 | 24 | 68 | 79 | (Day 42) | 16.2 | [symbol] 98.5 | 526.7 |
| | | 25 | MM | F | 6.0 | [symbol] 3 | 73 | 23 | 104 | [symbol] 13 | (Day 42) | [symbol] .7 | 712.1 | 349.8 |
| | | 26 | MM | F | [symbol] .0 | 258 | 64 | 26 | 194 | 178.9 | (Day 43) | −7.5 | 1027.4 | 578.2 |

| | | | | | | | | | | | | | M | |
|---|---|---|---|---|---|---|---|---|---|---|---|---|---|---|
| | | | K | | | | | | | | | | The sCD26 | |
| A | | | sCD26 | | | | L | | | | | | titer variation [symbol] | |
| Adminis- | | C | Titer (ng/ml) | | | | DPP4 Titer (μM/min) | | | | | | baseline(%) on | |
| tration | B | Patient | Day 1 [symbol] | | Day 2 [symbol] | | Day 1 | | Day 1 [symbol] | | Day 2 [symbol] | | Day 2 [symbol] Pre/Post | |
| Frequency | RECIST | NO. | Pre | Post | Pre | Post | Pre | Post | Pre | Post | Pre | Post | Day 2 [symbol] Pre | Day 2 [symbol] Post |
| Once | PD | 1 | 8 [symbol] 4 0 | 256. [symbol] | 870.1 | 237.0 | 14.5 | [symbol] 0 | 12.8 | 5 [symbol] 3 | 12.5 | 5.2 | 86.4 | 23.6 |
| per | | 2 | 1082.4 | 342.8 | [symbol] 12.6 | 255.9 | 17.3 | [symbol] 2 | 14.5 | 6 [symbol] 5 | 11.6 | 4. [symbol] | 61.9 | 19.5 |
| two | | 3 | 1094.2 | 45 [symbol] .0 | 07 [symbol] .2 | 334. [symbol] | 20.0 | 0 [symbol] 9 | 1 [symbol] .1 | [symbol] .2 | 12.7 | 6.0 | 57.4 | 21.9 |
| weeks | | 4 | 741.5 | 334.4 | [symbol] 27.3 | 2 [symbol] 7 [symbol] 7 | 9.5 | 4 [symbol] 0 | 11.0 | 5 [symbol] 9 | 11.0 | 5.1 | 110.0 | 40.5 |
| (Q2W) | | 5 | 418.6 | 347. [symbol] | [symbol] 53.6 | 398. [symbol] | 15.1 | 7 [symbol] 9 | 7. [symbol] | 6 [symbol] 8 | 11.8 | 7. [symbol] | 7 [symbol] .5 | 39.9 |
| | | 6 | 656.3 | 2 [symbol] 5.5 | 589.1 | 2 [symbol] 8.5 | 12.9 | [symbol] .3 | 10.3 | 5 [symbol] 8 | 9.0 | 5.4 | 67.2 | 32.9 |
| | | 7 | N.A. | N.A [symbol] | 416 [symbol] 7 | N [symbol] A [symbol] | 10 [symbol] 0 | 5.2 | N.A. | N.A [symbol] | [symbol] .5 | N.A [symbol] | 91.0 | N.A. |
| | | 8 | 61 [symbol] .3 | 338.7 | 476.9 | 228 [symbol] 0 | 14.1 | 6 [symbol] 7 | [symbol] .8 | [symbol] 5 | 7.4 | 4.7 | 49.7 | 23.7 |
| | | 9 | 363.8 | 190. [symbol] | 331.8 | 169 [symbol] 0 | 12. [symbol] | [symbol] | 8.5 | 3 [symbol] 8 | 6.1 | 3. [symbol] | 39.9 | 20.3 |
| | | 10 | 3 [symbol] 0.2 | 234.2 | 394 [symbol] 9 | 171.2 | 13. [symbol] | 7.9 | 9.1 | 5 [symbol] 4 | [symbol] .2 | 4.4 | 55.2 | 23.9 |
| | SD | 11 | 1185.5 | 377. [symbol] | 963.5 | 303.3 | 1 [symbol] | 7.9 | 16.3 | 7.3 | 13. [symbol] | 6.3 | 78.5 | 24.7 |
| | | 12 | 997 [symbol] 7 | 366.9 | 327.9 | 1 [symbol] .5 | 15 [symbol] 8 | 6.8 | 14. [symbol] | [symbol] .9 | 7.4 | 3.4 | 30 1 | 9.4 |
| | | 13 | 616.0 | 191.4 | 255.3. | 43. [symbol] | 1 [symbol] | 4. [symbol] | 0. [symbol] | 4.5 | 5.5 | 1.8 | 34.9 | 6.0 |
| | | 14 | 258.3 | 203.9 | 13 [symbol] .8 | 83.0 | 8.7 | 4.9 | [symbol] .7 | 5.2 | 4.0 | 2.7 | 34.5 | 20.9 |
| | | 15 | 423.3 | 300.6 | 318. [symbol] | 196.6 | 13.3 | 7 [symbol] 7 | 9.8 | 7 [symbol] 5 | 7.9 | 5.4 | 40.4 | 28.7 |
| | | 16 | [symbol] 70.4 | 448.0 | 522.5 | 149.7 | 17.3 | 8.5 | 10.8 | 7.2 | 9.3 | 3.5 | 4 [symbol] .9 | 12.0 |
| | | 17 | 454.9 | 219.7 | 390.1 | 288.3 | 1 [symbol] | 9.7 | 8.3 | 4.7 | [symbol] 2 | 5. [symbol] | 34.2 | 25.3 |
| | | 18 | 557.8 | 329.5 | 4 [symbol] 2.9 | 233.4 | 30.8 | 15.2 | 16.6 | 10.3 | 14. [symbol] | [symbol] .2 | 37.6 | 1 [symbol] .2 |
| Once | PD | 19 | 160.4 | N.A. | N.A. | N.A. | 7.8 | 3.2 | 2.8 | N.A. | N.A. | N.A. | N.A. | N.A. |
| a | | 20 | 317.7 | 2 [symbol] 8.2 | 215.8 | 191.2 | 10.6 | 5.8 | 4.6 | 3.9 | 2.9 | 2. [symbol] | 2 [symbol] .0 | 23.0 |
| week | | 21 | 199.0 | 164.0 | 177.2 | 161.6 | 9.7 | 5.6 | 3.4 | 2.7 | 2.4 | 2.7 | 24.6 | 22.4 |
| (Q1W) | SD | 22 | 250.7 | 275.7 | 227.3 | 234.8 | [symbol] | [symbol] .5 | 3. [symbol] | 3.7 | 2.5 | 2.7 | 16 [symbol] 8 | 17.4 |
| | | 23 | 149.3 | 72.0 | 113.3 | [symbol] 2.3 | 10.4 | 4.0 | 2.7 | 1.7 | 2. [symbol] | [symbol] .9 | 14.9 | 10. [symbol] |
| | | 24 | 237.8 | 252.4 | N.A. | N.A. | 11.2 | [symbol] .4 | 2.5 | 2.0 | N.A [symbol] | N.A [symbol] | N.A. | N.A. |
| | | 25 | 121.9 | 110.2 | 140.0 | 144.2 | 11.1 | 4. [symbol] | 2.2 | 2.4 | 3.0 | 2.5 | 19.7 | 20.2 |
| | | 26 | 285. [symbol] | 287.1 | 302.9 | 261.9 | 12.0 | [symbol] .5 | 2.6 | 3.0 | 2.2 | 2.8 | 29.5 | 25.5 |

Abbreviations: BMI = body mass index;
MM = malignant mesothelioma;
N.A. = not assessed;
PD = progressive disease;
PFS = progression free survival;
RCC = renal cell carcinoma;
SD = stable disease;
UTC = urothelial carcinoma; [symbol]
indicates data missing or illegible when filed

TABLE 4
Detailed information about 26 evaluable cases
MM (male/female)n = 19 (11/8)

| | Once every two weeks (Q2W) | | | | | One a week (Q1W) | | | |
|---|---|---|---|---|---|---|---|---|---|
| [symbol] | 0.1 | 0.4 | 1.0 | 2.0 | Total | 2.0 | 4.0 | 6.0 | Total |
| SD | —/1 | 1/— | —/— | 2/1 | 3/2 | —/2 | 1/— | —/2 | 1/4 |
| PD | 2/— | —/1 | 2/— | 2/— | 6/1 | —/— | 1/— | —/1 | 1/1 |
| Total | 2/1 | 1/1 | 2/— | 4/1 | 9/3 | —/2 | 2/1 | —/3 | 2/5 |

RCC (male/female)n = 6 (5/1)

| | Once every two weeks (Q2W) | | | | | One a week (Q1W) | | | |
|---|---|---|---|---|---|---|---|---|---|
| [symbol] | 0.1 | 0.4 | 1.0 | 2.0 | Total | 2.0 | 4.0 | 6.0 | Total |
| SD | —/— | 1/— | —/— | 1/1 | 2/1 | —/— | —/— | —/— | —/— |
| PD | —/— | —/— | 2/— | 1/— | 3/— | —/— | —/— | —/— | —/— |
| Total | —/— | 1/— | 2/— | 2/1 | 5/1 | —/— | —/— | —/— | —/— |

UTC (male female)n = 1 (—/1)

| | Once every two weeks (Q2W) | | | | | One a week (Q1W) | | | |
|---|---|---|---|---|---|---|---|---|---|
| [symbol] | 0.1 | 0.4 | 1.0 | 2.0 | Total | 2.0 | 4.0 | 6.0 | Total |
| SD | —/— | —/— | —/— | —/— | —/— | —/— | —/— | —/— | —/— |
| PD | —/— | —/— | —/— | —/— | —/— | —/1 | —/— | —/— | —/1 |
| Total | —/— | —/— | —/— | —/— | —/— | —/1 | —/— | —/— | —/1 |

Abbreviations: MM = malignant mesothelioma;
PD = progressive disease;
RCC = renal cell carcinoma;
SD = stable disease;
UTC = urothelial carcinoma

Since the number of cases in each antibody dose cohort was not sufficient for statistical analysis, in this study, a total of 26 cases were further classified according to 1) tumor tissue and 2) drug administration frequency, and whether or not serum soluble CD26 titer variation could be a prognostic biomarker for YS110 treatment was examined (see Table 3 for detailed information on these 26 cases).

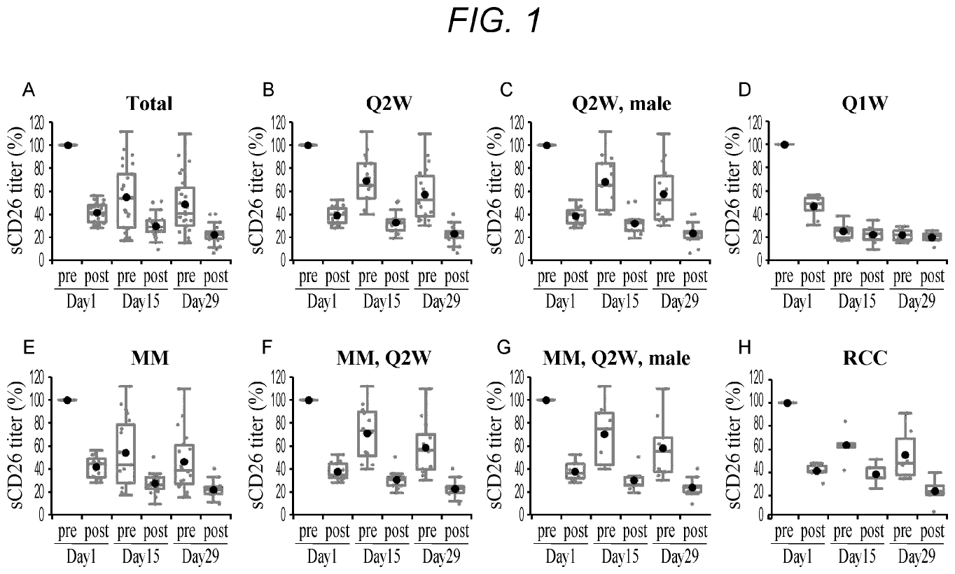

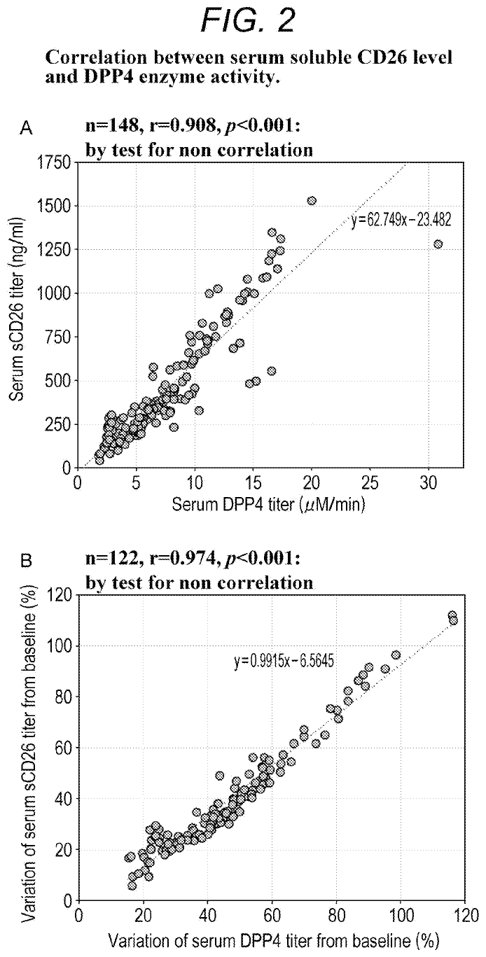

First, the serum soluble CD26 titer variation during YS110 treatment in each group was examined by boxplot analysis. Serum soluble CD26 titer decreased consistently immediately after YS110 administration on Day 1, Day 15 and Day 29, and gradually recovered until the next YS110 infusion, but did not return to the previous level before administration (FIG. 1A). This pattern was similarly observed in 18 cases treated with a Q2W drug administration schedule (FIG. 1B). In contrast, a clear difference was observed in 8 cases treated with a Q1W schedule. As shown in Table 3, a relatively higher dose (2 to 6 mg/kg) of antibody was administered in the Q1W case compared to the Q2W case (0.1 to 2 mg/kg). These differences in antibody dose and administration frequency greatly affected serum soluble CD26 titer before administration on Day 15 and Day 29 (FIG. 1D). Recovery of serum soluble CD26 titer after YS110 administration was not evident in Q1W cases with higher drug administration frequency. Q2W administration was performed in 14 male cases and 4 female cases, and Q1W administration was performed in 2 male cases and 6 female cases (Table 4). Distribution bias between male cases of Q2W and Q1W administration and female case of Q2W and Q1W administration was significant (p=0.026 by Fisher's exact test). Furthermore, the number of cases in the Q1W cohort (8 cases) was insufficient for additional statistical analysis. Therefore, a focus was mainly on Q2W cases and male cases for additional analysis. Initial decrease and subsequent recovery of serum soluble CD26 titer was similarly observed in both MM 19 cases and RCC 6 cases, including additional stratification (FIGS. 1C, F, and G) of the groups into the cohort that includes male 14 cases with Q2W administration, MM 12 cases with Q2W administration, and male and MM 9 cases with Q2W administration (FIGS. 1E and H). As shown in FIG. 2, the absolute value or titer variation of serum soluble CD26 titer was strongly correlated with the level of serum DPP4 enzyme activity (r=0.908, p<0.001 or r=0.974, p<0.001). Since YS110 does not directly inhibit DPP4 enzyme activity (Y's Therapeutics Inc. USA IND. 2008; 100657: Section 8, 8.2.1.5:289), the decrease in serum DPP4 enzyme activity after YS110 administration is due to a decrease in serum soluble CD26 protein levels.

### (8) Difference Between the Serum Soluble CD26/DPP4 Titer Variation Before Administration on Day 29 and the Tumor Volume Variation on Day 43 in SD Cohort and PD Cohort by Scatter Plot Analysis

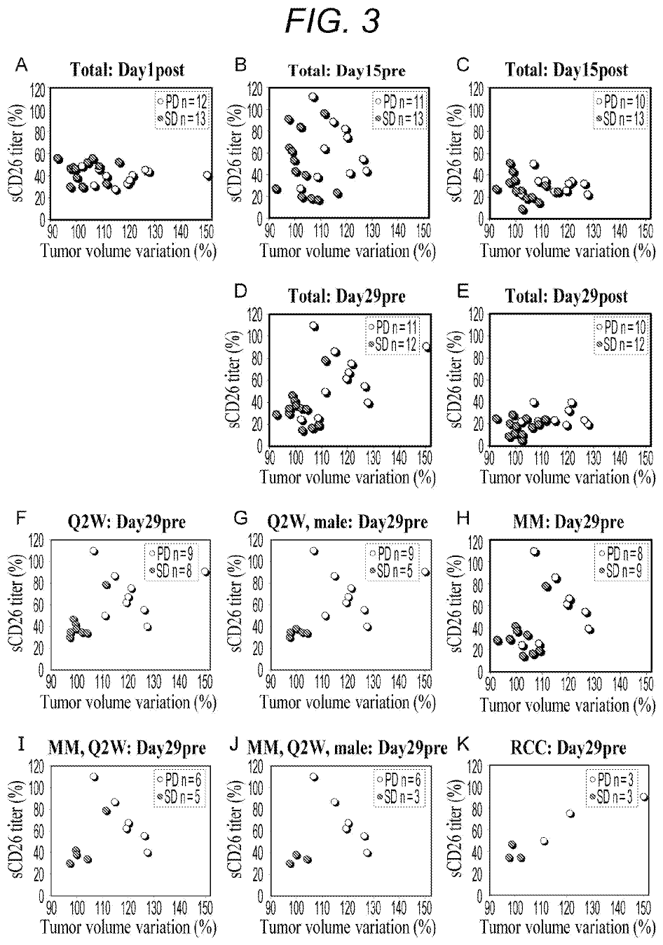

Next, by scatter plot analysis after the start of YS110 administration, a potential relationship between the serum soluble CD26 titer variation before/after administration on Day 1, Day 15, and Day 29 and the tumor volume variation on Day 43 was examined for a total of 25 cases stratified by the SD cohort and the PD cohort. The tumor volume variation in an SD group is naturally expected to be lower than in a PD group. Serum soluble CD26 titer decreased significantly in both SD cohort and PD cohort immediately after YS110 infusion on Day 1, Day 15, and Day 29 (FIGS. 3A, C, and E). On the other hand, a significant difference between the SD group and PD group in serum soluble CD26 titer variation was observed before infusion on Day 29. The serum soluble CD26 titer variation before infusion on day 29 of the SD cohort were at lower levels compared to the PD group (FIG. 3D). Furthermore, this phenomenon was clearly observed in each stratification group such as 17 cases with Q2W administration, 14 cases of males with Q2W administration, 18 cases of MM, 11 cases of MM with Q2W administration, 9 cases of male and MM with Q2W administration or 6 cases of RCC (FIGS. 3Ff to K, respectively). As these scatter plot analysis show, the serum soluble CD26 titer variation in the SD cohort is lower than in PD cases, as measured before YS110 administration, and this difference is particularly evident before Q2W administration on Day 29.

(9) Correlation Between the Serum Soluble CD26/DPP4 Titer Variation Before/after Administration on Day 29 and the Tumor Volume Variation and/or PFS by PPMC/SRDC Analysis

PPMC analysis and SRDC analysis were performed to examine the correlation between the serum soluble CD26 antibody titer variation before/after administration on Day 1, Day 15, and Day 29 and the tumor volume variation or PFS determined by RECIST criteria on Day 43 after YS110 administration. In the FIH phase I clinical study, 13 cases were determined to be SD and 13 cases were determined to be PD by RECIST, and among 13 SD cases, YS110 was particularly effective in 7 cases, and PFS exceeded 180 days (Table 5).

TABLE 5
Correlation between serum sCD26/DPP4 titer variation
and tumor volume change/PFS by PPMC or SRDC analysis

| | | | Peason's Product - | | Spearman's rank | |
|---|---|---|---|---|---|---|
| | | | m [symbol] ent correlation | | difference correlation | |
| Var. 1 | Var. 2 | n | r | P value | ρ | P value |
| tumor volume % change | PFS | 25 | −0.514 | 0.008 ** | −0.504 | 0.014 * |
| tumor volume % change | sCD26 Day 1 Post | 25 | −0.214 | 0.308 | −0.198 | 0.333 |
| ″ | sCD26 Day 15 Pre | 24 | −0.085 | 0.69 [symbol] | 0.002 | 0.993 |
| ″ | sCD26 Day 15 Post | 23 | −0.115 | 0.606 | −0.169 | 0.428 |
| ″ | sCD26 Day 29 Pre | 23 | 0.548 | 0.00 [symbol] * | 0.553 | 0.009 ** |
| ″ | sCD26 Day 29 Post | 22 | 0.358 | 0.102 | 0. [symbol] 04 | 0.163 |
| ″ | DPP4 Day 1 Post | 25 | −0.146 | 0.490 | −0.182 | 0.374 |
| ″ | DPP4 Day 15 Pre | 24 | −0.068 | 0.757 | −0.023 | 0.910 |
| ″ | DPP4 Day 15 Post | 23 | −0.039 | 0.862 | −0.037 | 0.864 |
| ″ | DPP4 Day 29 Pre | 23 | 0.502 | 0.014 | 0.531 | 0.013 * |
| ″ | DPP4 Day 29 Post | 22 | 0.379 | 0.082 | 0.451 | 0.039 * |
| PFS (days) | sCD26 Day 1 Post | 26 | 0.047 | 0.821 | 0.083 | 0.678 |
| ″ | sCD26 Day 15 Pre | 25 | −0.021 | 0.922 | −0.099 | 0.626 |
| ″ | sCD26 Day 15 Post | 24 | −0.010 | 0.964 | 0.017 | 0.935 |
| ″ | sCD26 Day 29 Pre | 24 | −0.351 | 0.0 [symbol] 3 | −0.205 | 0.325 |
| ″ | sCD26 Day 29 Post | 23 | −0.521 | 0.010 ** | −0.332 | 0.119 |
| ″ | DPP4 Day 1 Post | 26 | −0.109 | 0.600 | −0.048 | 0.809 |
| ″ | DPP4 Day 15 Pre | 25 | −0.022 | 0.919 | −0.089 | 0.663 |
| ″ | DPP4 Day 15 Post | 24 | −0.034 | 0.877 | −0.072 | 0.732 |
| ″ | DPP4 Day 29 Pre | 24 | −0.253 | 0.235 | −0.167 | 0.423 |
| ″ | DPP4 Day 29 Post | 23 | −0.442 | 0.034 * | −0.381 | 0.074 |

[symbol]

indicates data missing or illegible when filed

In a total of 25 cases, a statistically significant correlation was observed between the serum soluble CD26 titer variation before administration on Day 29 and the tumor volume variation on Day 43 (p=0.006 or p=0.009 by PPMC/SRDC (Table 5)). A statistically significant correlation was also observed between serum soluble CD26 titer variation and PFS (in a total of 26 cases, p=0.011 after administration on Day 29 by PPMC, (Table 5)). In addition, a statistically significant correlation was observed between serum titer variation in DPP4 enzyme activity and tumor volume or PFS as in the case of serum soluble CD26 titer (Table 5). Similarly, statistically significant correlations were observed between serum soluble CD26/DPP4 titer before administration and tumor volume on Day 29 and between serum soluble CD26/DPP4 titer before administration and/or after administration and PFS on Day 29 in 18 cases with Q2W administration frequency and 14 cases of male with Q2W administration frequency (Tables 6 and 7).

TABLE 6
Correlation between serum sCD26/DPP4 titer variation (%) and tumor volume change
(%)/PFS (days) in 18 cases with Q2W administration by PPMC or SRDC analysis

| | | | Peason's Product- | | Spearman's rank | |
|---|---|---|---|---|---|---|
| | | | moment correlation | | difference correlation | |
| Variation 1 | Variation 2 | n | r | P value | ρ | P value |
| tumor volume % change | PFS | 17 | −0.573 | 0.015 | −0.614 | 0.014 * |
| tumor volume % change | sCD26 Day 1 Post | 17 | −0.004 | 0.989 | −0.039 | 0.875 |
| ″ | sCD26 Day 15 Pre | 16 | −0.145 | 0.600 | −0.153 | 0.554 |
| ″ | sCD26 Day 15 Post | 16 | −0.344 | 0.196 | −0.415 | 0.108 |
| ″ | sCD26 Day 29 Pre | 17 | 0.513 | 0.034 * | 0.625 | 0.012 * |
| ″ | sCD26 Day 29 Post | 16 | 0.390 | 0.138 | 0.371 | 0.151 |
| ″ | DPP4 Day 1 Post | 17 | 0.090 | 0.735 | 0.029 | 0.906 |
| ″ | DPP4 Day 15 Pre | 16 | −0.184 | 0.501 | −0.218 | 0.399 |
| ″ | DPP4 Day 15 Post | 16 | −0.347 | 0.192 | −0.329 | 0.202 |
| ″ | DPP4 Day 29 Pre | 17 | 0.438 | 0.079 | 0.529 | 0.034 * |
| ″ | DPP4 Day 29 Post | 16 | 0.317 | 0.237 | 0.374 | 0.148 |
| PFS (days) | sCD26 Day 1 Post | 18 | 0.140 | 0.584 | 0.246 | 0.311 |
| ″ | sCD26 Day 15 Pre | 17 | −0.143 | 0.589 | −0.247 | 0.324 |
| ″ | sCD26 Day 15 Post | 17 | −0.001 | 0.996 | 0.141 | 0.574 |
| ″ | sCD26 Day 29 Pre | 18 | −0.523 | 0.025 * | −0.522 | 0.031 * |
| ″ | sCD26 Day 29 Post | 17 | −0.611 | 0.008 ** | −0.460 | 0.066 |
| ″ | DPP4 Day 1 Post | 18 | −0.033 | 0.899 | 0.309 | 0.203 |
| ″ | DPP4 Day 15 Pre | 17 | −0.134 | 0.613 | −0.166 | 0.506 |
| ″ | DPP4 Day 15 Post | 17 | −0.043 | 0.871 | −0.020 | 0.937 |
| ″ | DPP4 Day 29 Pre | 18 | −0.425 | 0.079 | −0.418 | 0.085 |
| ″ | DPP4 Day 29 Post | 17 | −0.580 | 0.013 * | −0.515 | 0.039 * |

TABLE 7
Correlation between serum sCD26/DPP4 titer variation (%) and tumor volume change
(%)/PFS (days) in 14 male cases with Q2W administration by PPMC or SRDC analysis

| | | | Peason's Product- | | Spearman's rank | |
|---|---|---|---|---|---|---|
| | | | moment correlation | | difference correlation | |
| Variation 1 | Variation 2 | n | r | P value | ρ | P value |
| tumor volume % change | PFS | 14 | −0.545 | 0.043 * | −0.540 | 0.051 |
| tumor volume % change | sCD26 Day 1 Post | 14 | 0.163 | 0.585 | 0.262 | 0.346 |
| ″ | sCD26 Day 15 Pre | 13 | −0.258 | 0.403 | −0.297 | 0.304 |
| ″ | sCD26 Day 15 Post | 13 | −0.261 | 0.399 | −0.225 | 0.435 |
| ″ | sCD26 Day 29 Pre | 14 | 0.505 | 0.065 | 0.648 | 0.019 * |
| ″ | sCD26 Day 29 Post | 13 | 0.406 | 0.174 | 0.445 | 0.123 |
| ″ | DPP4 Day 1 Post | 14 | 0.224 | 0.451 | 0.209 | 0.452 |
| ″ | DPP4 Day 15 Pre | 13 | −0.277 | 0.369 | −0.374 | 0.196 |
| ″ | DPP4 Day 15 Post | 13 | −0.287 | 0.350 | −0.231 | 0.424 |
| ″ | DPP4 Day 29 Pre | 14 | 0.440 | 0.117 | 0.591 | 0.033 * |
| ″ | DPP4 Day 29 Post | 13 | 0.337 | 0.267 | 0.451 | 0.119 |
| PFS (days) | sCD26 Day 1 Post | 14 | −0.301 | 0.302 | −0.011 | 0.968 |
| ″ | sCD26 Day 15 Pre | 13 | 0.066 | 0.834 | −0.143 | 0.619 |
| ″ | sCD26 Day 15 Post | 13 | −0.264 | 0.393 | −0.044 | 0.879 |
| ″ | sCD26 Day 29 Pre | 14 | −0.552 | 0.040 * | −0.553 | 0.046 * |
| ″ | sCD26 Day 29 Post | 13 | −0.712 | 0.005 ** | −0.546 | 0.059 |
| ″ | DPP4 Day 1 Post | 14 | −0.290 | 0.322 | 0.229 | 0.408 |
| ″ | DPP4 Day 15 Pre | 13 | 0.050 | 0.874 | −0.036 | 0.901 |
| ″ | DPP4 Day 15 Post | 13 | −0.211 | 0.499 | −0.108 | 0.709 |
| ″ | DPP4 Day 29 Pre | 14 | −0.451 | 0.107 | −0.474 | 0.087 |
| ″ | DPP4 Day 29 Post | 13 | −0.672 | 0.010 * | −0.654 | 0.024 * |

In 19 cases of MM, a statistically significant correlation was observed between serum DPP4 titer variation before/after administration and tumor volume on Day 29 by SRDC analysis, and the correlation between the serum soluble CD26 titer before administration on Day 29 and the tumor volume reached almost statistically significant by PPMC analysis (p=0.065). A statistically significant correlation was observed between serum soluble CD26 titer after administration on Day 29 and PFS by PPMC analysis, and the correlation between serum DPP4 titer after administration on Day 29 and PFS reached almost statistically significant (p=0.056 in PPMC analysis, p=0.069 in SRDC analysis) (Table 8).

TABLE 8
Correlation between serum sCD26/DPP4 titer variation (%) and tumor volume
change (%)/PFS (days) in 19 MM cases by PPMC or SRDC analysis

| | | | Peason's Product- | | Spearman's rank | |
|---|---|---|---|---|---|---|
| | | | moment correlation | | difference correlation | |
| Variation 1 | Variation 2 | n | r | P value | ρ | P value |
| tumor volume % change | PFS | 18 | −0.668 | 0.002 ** | −0.568 | 0.019 * |
| tumor volume % change | sCD26 Day 1 Post | 18 | −0.147 | 0.566 | −0.164 | 0.499 |
| ″ | sCD26 Day 15 Pre | 18 | 0.193 | 0.450 | 0.201 | 0.407 |
| ″ | sCD26 Day 15 Post | 18 | 0.030 | 0.908 | −0.038 | 0.875 |
| ″ | sCD26 Day 29 Pre | 17 | 0.388 | 0.125 | 0.461 | 0.065 |
| ″ | sCD26 Day 29 Post | 17 | 0.290 | 0.265 | 0.336 | 0.179 |
| ″ | DPP4 Day 1 Post | 18 | 0.060 | 0.817 | −0.040 | 0.868 |
| ″ | DPP4 Day 15 Pre | 18 | 0.230 | 0.365 | 0.207 | 0.392 |
| ″ | DPP4 Day 15 Post | 18 | 0.135 | 0.599 | 0.137 | 0.572 |
| ″ | DPP4 Day 29 Pre | 17 | 0.370 | 0.146 | 0.515 | 0.040 * |
| ″ | DPP4 Day 29 Post | 17 | 0.371 | 0.145 | 0.593 | 0.018 * |
| PFS (days) | sCD26 Day 1 Post | 19 | 0.100 | 0.688 | 0.137 | 0.563 |
| ″ | sCD26 Day 15 Pre | 19 | −0.150 | 0.546 | −0.134 | 0.570 |
| ″ | sCD26 Day 15 Post | 19 | 0.021 | 0.934 | −0.056 | 0.811 |
| ″ | sCD26 Day 29 Pre | 18 | −0.328 | 0.187 | −0.176 | 0.467 |
| ″ | sCD26 Day 29 Post | 18 | −0.527 | 0.023 * | −0.354 | 0.145 |
| ″ | DPP4 Day 1 Post | 19 | −0.138 | 0.578 | −0.122 | 0.604 |
| ″ | DPP4 Day 15 Pre | 19 | −0.153 | 0.537 | −0.140 | 0.553 |
| ″ | DPP4 Day 15 Post | 19 | −0.080 | 0.748 | −0.179 | 0.448 |
| ″ | DPP4 Day 29 Pre | 18 | −0.251 | 0.320 | −0.210 | 0.386 |
| ″ | DPP4 Day 29 Post | 18 | −0.457 | 0.056 | −0.441 | 0.069 |

In 12 cases of MM with Q2W administration frequency, no statistically significant correlation was observed between the serum soluble CD26/DPP4 titer variation and the tumor volume. The correlation between serum soluble CD26/DPP4 titer after administration on Day 29 and PFS reached statistically significant (Table 9).

TABLE 9
Correlation between serum sCD26/DPP4 titer variation (%) and tumor volume change
(%)/PFS (days) in 12 MM cases with Q2W administration by PPMC or SRDC analysis

| | | | Peason's Product- | | Spearman's rank | |
|---|---|---|---|---|---|---|
| | | | moment correlation | | difference correlation | |
| | | | (PPMC) | | (SRDC) | |
| Variation 1 | Variation 2 | n | r | P value | ρ | P value |
| tumor volume % change | PFS | 11 | −0.730 | 0.009 ** | −0.782 | 0.013 * |
| tumor volume % change | sCD26 Day 1 Post | 11 | 0.051 | 0.885 | 0.082 | 0.796 |
| ″ | sCD26 Day 15 Pre | 11 | −0.089 | 0.802 | −0.045 | 0.886 |
| ″ | sCD26 Day 15 Post | 11 | −0.208 | 0.550 | −0.309 | 0.328 |
| ″ | sCD26 Day 29 Pre | 11 | 0.224 | 0.520 | 0.391 | 0.216 |
| ″ | sCD26 Day 29 Post | 11 | 0.326 | 0.339 | 0.418 | 0.186 |
| ″ | DPP4 Day 1 Post | 11 | 0.224 | 0.520 | 0.082 | 0.796 |
| ″ | DPP4 Day 15 Pre | 11 | −0.079 | 0.822 | −0.118 | 0.709 |
| ″ | DPP4 Day 15 Post | 11 | −0.182 | 0.603 | −0.209 | 0.509 |
| ″ | DPP4 Day 29 Pre | 11 | 0.116 | 0.741 | 0.364 | 0.250 |
| ″ | DPP4 Day 29 Post | 11 | 0.249 | 0.471 | 0.482 | 0.128 |
| PFS (days) | sCD26 Day 1 Post | 12 | 0.298 | 0.356 | 0.312 | 0.301 |
| ″ | sCD26 Day 15 Pre | 12 | −0.309 | 0.338 | −0.262 | 0.384 |
| ″ | sCD26 Day 15 Post | 12 | 0.097 | 0.770 | 0.142 | 0.638 |
| ″ | sCD26 Day 29 Pre | 12 | −0.529 | 0.077 | −0.532 | 0.078 |
| ″ | sCD26 Day 29 Post | 12 | −0.653 | 0.019 * | −0.539 | 0.074 |
| ″ | DPP4 Day 1 Post | 12 | 0.034 | 0.918 | 0.340 | 0.259 |
| ″ | DPP4 Day 15 Pre | 12 | −0.320 | 0.320 | −0.192 | 0.525 |
| ″ | DPP4 Day 15 Post | 12 | −0.061 | 0.854 | −0.064 | 0.832 |
| ″ | DPP4 Day 29 Pre | 12 | −0.446 | 0.150 | −0.525 | 0.082 |
| ″ | DPP4 Day 29 Post | 12 | −0.629 | 0.026 * | −0.624 | 0.038 * |

In 9 cases of male and MM treated with Q2W administration, no significant difference was observed in the variation between serum soluble CD26/DPP4 titer and tumor volume, but there was a trend of correlation between serum soluble CD26/DPP4 titer before/after administration on Day 29 and PFS (Table 10).

TABLE 10
Correlation between serum sCD26/DPP4 titer variation (%) and tumor volume change (%)/PFS
(days) in 9 MM, male cases with Q2W administration by PPMC or SRDC analysis

| | | | Peason's Product- | | Spearman's rank | |
|---|---|---|---|---|---|---|
| | | | moment correlation | | difference correlation | |
| Variation 1 | Variation 2 | n | r | P value | ρ | P value |
| tumor volume % change | PFS | 9 | −0.795 | 0.008 ** | −0.748 | 0.034 * |
| tumor volume % change | SCD26 Day 1 Post | 9 | 0.208 | 0.605 | 0.317 | 0.370 |
| ″ | sCD26 Day 15 Pre | 9 | −0.186 | 0.645 | −0.150 | 0.671 |
| ″ | sCD26 Day 15 Post | 9 | −0.141 | 0.728 | −0.150 | 0.671 |
| ″ | sCD26 Day 29 Pre | 9 | 0.176 | 0.663 | 0.417 | 0.239 |
| ″ | sCD26 Day 29 Post | 9 | 0.208 | 0.605 | 0.333 | 0.346 |
| ″ | DPP4 Day 1 Post | 9 | 0.257 | 0.519 | 0.117 | 0.741 |
| ″ | DPP4 Day 15 Pre | 9 | −0.166 | 0.682 | −0.267 | 0.451 |
| ″ | DPP4 Day 15 Post | 9 | −0.165 | 0.683 | −0.117 | 0.741 |
| ″ | DPP4 Day 29 Pre | 9 | 0.071 | 0.861 | 0.383 | 0.278 |
| ″ | DPP4 Day 29 Post | 9 | 0.123 | 0.762 | 0.400 | 0.258 |
| PFS (days) | sCD26 Day 1 Post | 9 | −0.070 | 0.865 | 0.084 | 0.812 |
| ″ | sCD26 Day 15 Pre | 9 | −0.172 | 0.671 | −0.252 | 0.476 |
| ″ | sCD26 Day 15 Post | 9 | −0.121 | 0.765 | −0.008 | 0.981 |
| ″ | sCD26 Day 29 Pre | 9 | −0.600 | 0.090 | −0.664 | 0.060 |
| ″ | sCD26 Day 29 Post | 9 | −0.626 | 0.072 | −0.571 | 0.106 |
| ″ | DPP4 Day 1 Post | 9 | −0.065 | 0.873 | 0.336 | 0.342 |
| ″ | DPP4 Day 15 Pre | 9 | −0.201 | 0.617 | −0.118 | 0.739 |
| ″ | DPP4 Day 15 Post | 9 | −0.152 | 0.707 | −0.143 | 0.686 |
| ″ | DPP4 Day 29 Pre | 9 | −0.510 | 0.168 | −0.647 | 0.067 |
| ″ | DPP4 Day 29 Post | 9 | −0.568 | 0.114 | −0.672 | 0.057 |

In 6 cases and 8 cases of RCC treated with Q2W and Q1W administration, the number of cases was not sufficient for PPMC/SRDC statistical analysis. From the above, a correlation was observed in the variation between serum soluble CD26/DPP4 titer before/after administration on Day 29 (before/after the third YS110 administration) and tumor volume or PFS. Although the number of cases in each stratified cohort was limited, it is considered to be important that a statistically significant difference was observed particularly in 18 cases and 14 cases of male treated with Q2W administration.

(10) Serum Soluble CD26/DPP4 Titer Before Administration on Day 29 of SD Cohort by Bar Graph Analysis (Significantly Lower than PD Cohort)

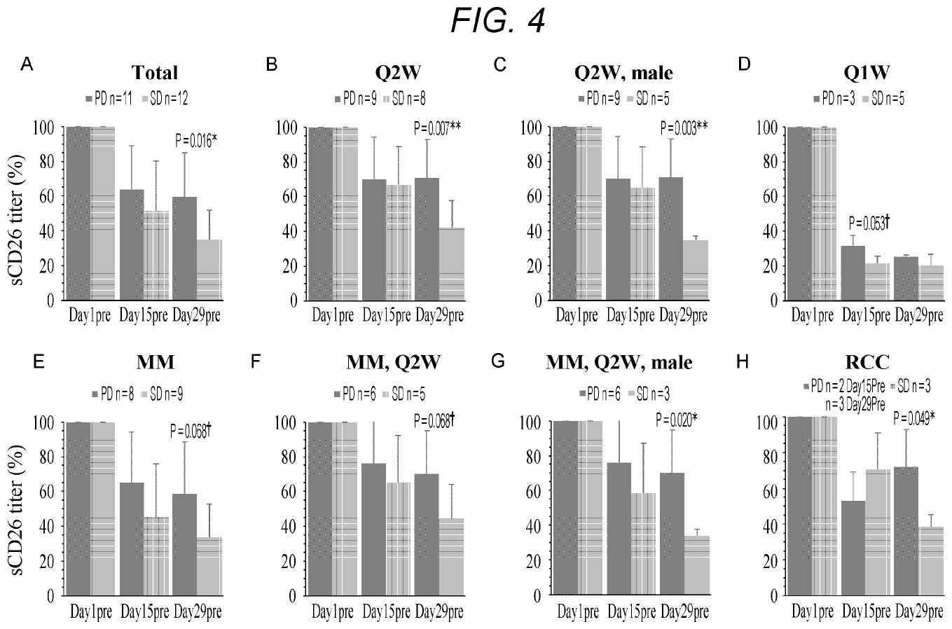

Based on the scatter diagram and PPMC/SRDC analysis, bar graph analysis of the serum soluble CD26 titer variation before administration on Day 1, Day 15, and Day 29 in SD cases and PD cases was performed. In a total of 23 cases (12 cases of SD and 11 cases of PD), serum soluble CD26 titer of the SD cohort and the PD cohort decreased from Day 1 before administration to Day 29 before administration. Of note, the serum soluble CD26 titer variation before administration on Day 29 in SD cases was significantly lower than in PD cases (p=0.016) (FIG. 4A). Similar results were observed in each stratified group, such as 17 cases treated with Q2W administration (p=0.007), 17 cases of MM (p=0.068), 11 cases of MM treated with Q2W administration (p=0.068), 9 cases of male and MM treated with Q2W administration (p=0.020), or 6 cases of RCC (p=0.049) (FIGS. 4B and E to H). A statistically significant difference between the SD cohort with the minimal p-value and the PD cohort were observed in 14 cases of male treated with Q2W administration (p=0.003) (FIG. 4C). In 8 cases treated with Q1W administration, the serum soluble CD26 titer variation in SD cases was lower than in PD cases and moved towards statistical significance before the third YS110 administration on Day 15 (p=0.053). This timing represents the same sample collection timing to evaluate serum soluble CD26 titer before administration on Day 29 in a Q2W treatment schedule (FIG. 4D).

### (11) Ability to Predict Serum Soluble CD26/DPP4 Titer Variation on SD or PFS Results by ROC Analysis in Stratified Groups

ROC analysis was employed to examine a cutoff titer (an index) of the serum soluble CD26/DPP4 titer variation before/after YS110 administration on Day 29 for SD and PFS≥90 days or ≥180 days results. Probability was evaluated with Fisher's exact test (Table 11).

TABLE 11
ROC analysis

| | | | | | | | | | | | | MM, Q2W, | |
|---|---|---|---|---|---|---|---|---|---|---|---|---|---|
| Column No. | | Total | | Q2W | | Q2W, male | | MM | | MM, Q2W | | male | |
| Cases of analysis for SD or | | 23 cases | | 17 cases (SD) | | Male 14 cases | | Malignant | | Malignant | | Malignant | |
| PFS Outcome | | (SD/PFS) | | 18 cases (PFS) | | (SD/PFS) | | Mesothelioma | | Mesothelioma | | Mesothelioma | |
| | | | | | | | | 17 cases (SD) | | 11 cases (SD) | | Male 9 cases | |
| | | | | | | | | 18 cases (PFS) | | 12 cases (PFS) | | (SD/PFS) | |
| Cases with | Cases with Q2W | 17 | | 17 or 18 | | 14 | | 11 or 12 | | 11 or 12 | | 9 | |
| Administration | Cases with Q1W | 6 | | 0 | | 0 | | 6 | | 0 | | 0 | |
| Frequency | | | | | | | | | | | | | |
| Outcome: SD | sCD26 measured point | Day 29 Pre | | Day 29 Pre | | Day 29 Pre | | Day 29 Post | | Day 29 Post | | Day 29 Pre | |
| Index: Cut-off | Index: Cut-off titer(%) | 46.4 | | 46.4 | | 37.7 | | 18.2 | | 18.2 | | 37.7 | |
| titer with | AUC | 0.795 | | 0.888 | | 1.000 | | 0.736 | | 0.733 | | 1.000 | |
| variation of | Sensitivity(%) | 91.7 | (11/12) | 87.5 | (7/8) | 100.0 | (5/5) | 55.6 | (5/9) | 60.0 | (3/5) | 100.0 | (3/3) |
| serum sCD26(%) | 1 [symbol] Specificity(%) | 27.3 | (3/11) | 11.1 | (1/9) | 0.0 | (0/9) | 0.0 | (0/8) | 0.0 | (0/6) | 0.0 | (0/6) |
| titer from | Fisher's Exact Test | P = 0.003 ** | | P = 0.003 ** | | P < 0.001 ** | | P = 0.029 * | | P = 0.061 * | | P = 0.012 * | |
| baseline | PPV | 0.786 | | 0.875 | | 1.000 | | 1.000 | | 1.000 | | 1.000 | |
| | NPV | 0.889 | | 0.889 | | 1.000 | | 0.667 | | 0.750 | | 1.000 | |
| Outcome: | sCD26 measured point | Day 29 Post | | Day 29 Pre | | Day 29 Pre | | Day 29 Post | | Day 29 Post | | Day 29 Pre | |
| PFS > 90 | Index: Cut-off titer(%) | 18.2 | | 46.4 | | 37.7 | | 18.2 | | 18.2 | | 37.7 | |
| Index: Cut-off | AUC | 0.692 | | 0.917 | | 0.950 | | 0.708 | | 0.812 | | 1.000 | |
| titer with | Sensitivity(%) | 62.5 | (5/8) | 100.0 | (6/6) | 100.0 | (4/4) | 66.7 | (4/6) | 75.0 | (3/4) | 100.0 | (3/3) |
| variation of | 1 [symbol] Specificity(%) | 6.7 | (1/15) | 16.7 | (2/12) | 10.0 | (1/10) | 8.3 | (1/12) | 0.0 | (0/8) | 0.0 | (0/6) |
| serum sCD26(%) | Fisher's Exact Test | P = 0.009 ** | | P = 0.002 ** | | P < 0.001 ** | | P = 0.022 * | | P = 0.018 * | | P = 0.012 * | |
| titer from | PPV | 0.833 | | 0.750 | | 0.800 | | 0.800 | | 1.000 | | 1.000 | |
| baseline | NPV | 0.824 | | 1.000 | | 1.000 | | 0.846 | | 0.889 | | 1.000 | |
| Outcome: | sCD26 measured point | Day 29 Post | | Day 29 Pre | | Day 29 Pre | | Day 29 Post | | Day 29 Post | | Day 29 Pre | |
| PFS > 180 | Index: Cut-off titer(%) | 18.2 | | 46.4 | | 37.7 | | 18.2 | | 18.2 | | 37.7 | |
| Index: Cut-off | AUC | 0.759 | | 0.846 | | 0.879 | | 0.815 | | 1.000 | | 0.929 | |
| titer with | Sensitivity(%) | 71.4 | (5/7) | 100.0 | (5/5) | 100.0 | (3/3) | 80.0 | (4/5) | 100.0 | (3/3) | 100.0 | (2/2) |
| variation of | 1 [symbol] Specificity(%) | 6.3 | (1/16) | 23.1 | (3/13) | 18.2 | (2/11) | 7.7 | (1/13) | 0.0 | (0/9) | 14.3 | (1/7) |

| serum sCD26(%) | Fisher's Exact Test | P = 0.003 ** | P = 0.007 ** | P = 0.027 * | P = 0.008 ** | P = 0.005 ** | P = 0.083 † |
|---|---|---|---|---|---|---|---|
| titer from | PPV | 0.833 | 0.625 | 0.600 | 0.800 | 1.000 | 0.667 |
| baseline | NPV | 0.882 | 1.000 | 1.000 | 0.923 | 1.000 | 1.000 |

[symbol]

indicates data missing or illegible when filed

A total of 23 cases were examined to examine the index (46.4% or 18.2%) for SD and PFS≥90 or ≥180 days results, with statistically significant results (p=0.003 for SD, 0.005 or 0.003 for PFS, area under the curve (AUC) 0.795, 0.697 or 0.759, respectively) observed (Table 11; column total). For the columns of Q2W (17 or 18 cases), male with Q2W (14 cases), MM (17 or 18 cases), MM with Q2W (11 or 12 cases), MM and male with Q2W (9 cases), the index for each column of results was determined to be statistically significant or to tend to be statistically significant. In particular, in the column of male with Q2W (14 cases), the index 37.7% before YS110 administration on Day 29 for an outcome SD was statistically significant (p<0.001, AUC 1.000). Further, the index 37.7% for results PFS≥90 days or ≥180 days was statistically significant (p<0.001 or p=0.027, AUC 0.950 or 0.879, respectively). Taken together, it is demonstrated that the serum soluble CD26/DPP4 titer variation is a potential prognostic biomarker for YS110 antitumor therapy, particularly at a point of time just before/immediately after the third YS110 infusion on Day 29 of the Q2W dosing schedule, by the analysis on the serum soluble CD26/DPP4 titer variation during the YS110 treatment process.

### (12) Decrease of Soluble CD26 Levels in Culture Supernatants of CD26 Expressing MM Cell Lines and Non-Tumor Cells by Addition of Humanized Anti-CD26 Monoclonal Antibody

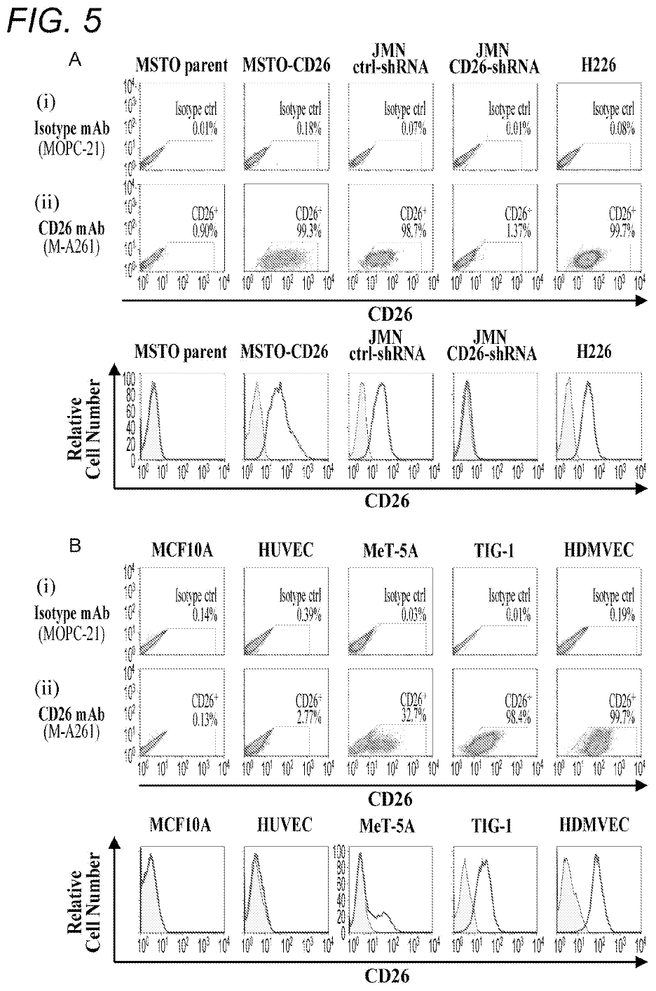

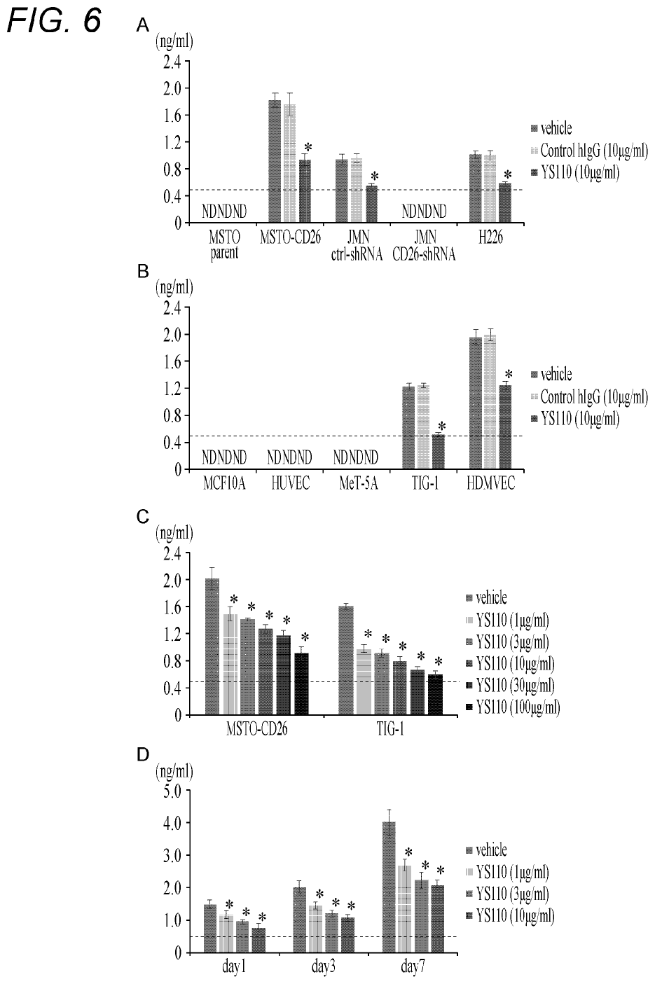

In the phase I study, an in vitro effect of YS110 on soluble CD26 production from MM cell lines was examined, as serum concentrations of soluble CD26 were significantly decreased after YS110 treatment in patients with CD26-expressing tumor (FIG. 1). To this end, various human CD26 positive or negative MM cell lines were selected. The MSTO parent was an endogenous human CD26 deficient cell line, whereas MSTO-CD26 stably expressed full-length human CD26. Stable shRNA knockdown of CD26 in JMN, an endogenous human CD26 positive cell line, significantly decreased CD26 expression compared to JMNctrl-shRNA cells (Yamazaki H, et al., Biochem Biophys Res Commun. 2012; 419(3): 529-36). Cell surface expression of CD26 in MM cell lines was shown in FIG. 5. First, the amount of soluble CD26 contained in the culture supernatant from 3 days of culture of CD26 positive or negative cells was measured. While soluble CD26 could be quantified in the culture supernatants of CD26 positive MSTO-CD26, JMNctrl-shRNA, and H226 cells, no soluble CD26 could be detected in the culture supernatants of CD26 negative MSTO parents and JMNCD26 shRNA cells regardless of YS110 treatment (FIG. 6A). Treatment with YS110 clearly reduced the amount of soluble CD26 in the culture supernatants of MSTO-CD26, JMNctrl-shRNA and H226 cells as compared to cells incubated with vehicle or control human IgG (FIG. 6A). Next, the production of soluble CD26 from non-tumor (normal) cells was examined. CD26 was clearly expressed on the cell surface of HDMVEC and TIG-1, while CD26 was hardly expressed in HUVEC and MCF10A, and partially expressed in MeT-5A (FIG. 5B). Soluble CD26 could be quantified in the culture supernatant of CD26 positive TIG-1 and HDMVEC cells, but soluble CD26 could not be detected in the culture supernatant of CD26 negative or low MCF10A, HUVEC and MeT-5A cells (FIG. 6B). Similar to the results shown in FIG. 6A, YS110 treatment clearly reduced the amount of soluble CD26 in the culture supernatant of TIG-1 and HDMVEC cells compared to cells incubated with vehicle or control human IgG (FIG. 6B). Treatment with YS110 reduced the production of soluble CD26 from both MSTO-CD26 and TIG-1 cells in a dose-dependent manner (FIG. 6C). Subsequent time course analysis showed that soluble CD26 levels in the supernatant of 3 days culture of MSTO-CD26 cells were slightly increased compared to 1 day culture of MSTO-CD26 cells, and increased soluble CD26 levels were observed in the supernatant of 7 days culture of MSTO-CD26 cells (FIG. 6D). A decrease in soluble CD26 levels after YS110 treatment was consistently observed in any culture period. Taken together, these data showed that soluble CD26 was produced from both CD26 positive tumor cells and non-tumor cells and that the addition of YS110 reduced soluble CD26 production from these cells in an antibody dose-dependent manner. It is believed that these in vitro effects are reflected in the significant decrease in serum soluble CD26 levels in patients with CD26-expressing tumor after YS110 administration.

A similar experiment was performed by administering 6 mg/kg once a week. As a result, the serum soluble CD26/DPP4 titer before/after YS110 administration on Day 2 was 48.64% for SD and 55.46% for PD, given the CD26/DPP4 titer before the first (Day 1, the same applies hereinafter) administration was 100% (p=0.026). Further, the serum soluble CD26/DPP4 titer on Day 15 pre (before administration) was 25.51% for SD and 33.47% for PD, given the CD26/DPP4 titer before the first administration was 100%. Further, the serum soluble CD26/DPP4 titer on Day 29 pre (before administration) was 25.10% for SD and 31.74% for PD, given the CD26/DPP4 titer before the first administration was 100%. Further, the serum soluble CD26/DPP4 titer on Day 15 post (after administration) was 24.06% for SD and 30.56% for PD, given the CD26/DPP4 titer before the first administration was 100%. Further, the serum soluble CD26/DPP4 titer on Day 29 post (after administration) was 25.64% for SD and 30.93% for PD, given the CD26/DPP4 titer before the first administration was 100%.

In this study, a scatter plot analysis for the relationship between the serum soluble CD26/DPP4 titer variation and the tumor volume variation according to RECIST response criteria suggested that a predictable period during a YS110 treatment process could be used to distinguish between SD cases and PD cases. This predictable period was before/after the third YS110 administration on Day 29 in the Q2W treatment schedule, and the results were found to be statistically significant by PPMC/SRDC analysis and bar graph analysis. ROC analysis defined the cutoff titer of the serum soluble CD26/DPP4 titer variation before/after administration on Day 29 as an index for the outcome of SD cases or PFS cases longer than 90 or 180 days, resulting in a significantly viable prediction under the obtained index. In particular, ROC analysis of 14 cases of male treated with the Q2W schedule defined a cut-off value of p<0.001 (Table 11). Similar results were obtained in 9 cases of male and MM treated with the Q2W dosing schedule (Table 11). The results were statistically significant despite the small number of cases in the stratification group, which strongly suggests that the serum soluble CD26/DPP4 titer variation is a definitive prognostic biomarker for cancer patients treated with YS110. Unlike the situation of the Q2W schedule, in cases treated with the Q1W schedule, the number of cases was not sufficient for analysis. However, the serum soluble CD26 titer variation before administration on Day 15 rather than before administration on Day 29 could be used to distinguish between PD cases and SD cases with a trend towards statistical significance (p=0.053), as shown in FIG. 4D. These data suggest that an increase in drug administration frequency and dosage (Q1W at YS110 dose levels 2, 4, 6 mg/kg) affects the optimal timing of serum soluble CD26 titer measurement, which may vary depending on the administration frequency and/or dosage of YS110.

As our robust in vitro and in vivo data show, YS110 induced cytolysis of MM cells via antibody-dependent cell-mediated cytotoxicity (ADCC), in addition to further inducing its direct antitumor effect via induction of cell cycle arrest in S/G1 phase (Inamoto T, et al., Clin Cancer Res. 2007; 13(14): 4191-200, Hayashi M, et al., Cancer Cell Int. 2016; 16:35). Another important mechanism of action of YS110 was nuclear translocation of CD26 molecules by internalization of a CD26-YS110 complex from the cell surface to inhibit growth of MM cells via suppression of expression of POLR2A gene, a component of RNA polymerase II. However, in CD26 expressing non-neoplastic cells such as human embryonic kidney HEK293 cells and normal T lymphocytes, the CD26-YS110 complex did not migrate into the nucleus (Yamada K, et al., PLOS One. 2013; 8(4): e62304, Hayashi M, et al., Cancers (Basel). 2019; 11(8): 1138). Furthermore, internalization of a CD26 antibody complex was dependent on the epitope of CD26 recognized by the specific monoclonal antibody. Internalization of CD26 was not observed from the cell surface of MM cells treated with mouse anti-human CD26 monoclonal antibody 5F8, recognized a different epitope of CD26 from that recognized by YS110, and showed no antitumor activity (Yamada K, et al., PLOS One. 2013; 8(4): e62304, Hatano R, et al., Diagn Pathol. 2014; 9:30).

Residues 201 to 211, 730, and 740 of CD26, together with a serine catalytic site of residue 630 constituting a CD26/DPPIV pocket structure, are essential for DPP4 enzymatic activity [26]. In contrast, YS110 recognizes an aa region from position 248 to 358 of CD26, which is different from the catalytic site (Hatano R, et al., (2014) supra, Dong R P, et al., Mol Immunol. 1998; 35(1): 13-11 21), and the binding of YS110 does not directly affect DPP4 enzymatic activity (Y's Therapeutics Inc. USA IND. 2008; 100657: Section 8, 8.2.1.5:289). Our current data showed that YS110 treatment reduced the production of soluble CD26 from both CD26 expressing MM cell lines and non-tumor cells (FIG. 6). A soluble form of CD26 begins at aa residue of position 39 and lacks cytoplasmic and transmembrane regions (Iwaki-Egawa S, et al., J Biochem. 14 1998; 124(2): 428-33), but the exact mechanism involved in the production and release of soluble CD26 from the cell surface has not yet been fully elucidated. Reduced soluble CD26 production following YS110 treatment may be due to antibody-mediated internalization of cell surface CD26 molecules (Yamada K, et al., (2013), supra). In a phase I clinical study using YS110, the serum concentration of soluble CD26 immediately after YS110 administration on Day 1 (after administration on Day 1) was significantly decreased as compared to the serum concentration before YS110 administration (before administration on Day 1) (FIG. 1). Fc receptor mediated phagocytosis of soluble CD26-YS110 complex by phagocytes may possibly be involved in rapid decrease of serum soluble CD26 after YS110 administration. In this study, it is demonstrated that while persisting low levels of serum soluble CD26/DPP4 titer after YS110 administration are commonly observed in SD cases compared to PD cases, no significant difference is observed in serum soluble CD26/DPP4 levels (on Day 1 after administration, on Day 15 after administration, on Day 29 after administration) immediately after YS110 administration between SD cases and PD cases (FIGS. 1 and 3).

The present results are the first findings showing that the serum soluble CD26/DPP4 titer variation in an early stage of treatment with the humanized anti-CD26 antibody YS110 may be a predictive biomarker of antitumor activity for CD26+ cancer patients including MM.

1. A method for selecting a cancer patient that have a potential to obtain a therapeutic effect with an anti-CD26 antibody, the method comprising:
comparing a level of soluble CD26 in serum of the patient before a reference administration of an anti-CD26 antibody with the level of soluble CD26 in the serum of the patient on a measurement date; and
when the level of soluble CD26 in the serum of the patient on the measurement date is less than 85% of the level of soluble CD26 in the serum of the patient before the reference administration, selecting the patient as having the potential to obtain a therapeutic effect with the anti-CD26 antibody, wherein
the measurement date is Day 1 to Day 60 with a reference administration date as Day 1, and
the patient is a patient to whom the anti-CD26 antibody is administered at least once as a reference administration, and
when the measurement date corresponds to an administration date of the anti-CD26 antibody, the level of soluble CD26 in the serum of the patient on the measurement date is the level of soluble CD26 in the serum of the patient before the administration of the anti-CD26 antibody on the measurement date.

2. The method according to claim 1, wherein when the measurement date is Day 1 to Day 30 from the reference administration, and the level of soluble CD26 in the serum of the patient on the measurement date is less than 60% of the level of soluble CD26 in the serum of the patient before the reference administration, the patient is selected as having the potential to obtain a therapeutic effect with the anti-CD26 antibody.

3. The method according to claim 1, wherein when the measurement date is Day 22 to Day 30 from the reference administration, and the level of soluble CD26 in the serum of the patient on the measurement date is less than 60% of the level of soluble CD26 in the serum of the patient before the reference administration, the patient is selected as having the potential to obtain a therapeutic effect with the anti-CD26 antibody.

4. The method according to claim 1, wherein when the measurement date is Day 2 to Day 8 from the reference administration, and the level of soluble CD26 in the serum of the patient on the measurement date is less than 50% of the level of soluble CD26 in the serum of the patient before the reference administration, the patient is selected as having the potential to obtain a therapeutic effect with the anti-CD26 antibody.

5. The method according to claim 1, wherein
when the patient is a patient to whom the anti-CD26 antibody is administered with a frequency of once every two weeks, and
the measurement date is Day 22 to Day 30 from the reference administration, and
the level of soluble CD26 in the serum of the patient on the measurement date is less than 65% of the level of soluble CD26 in the serum of the patient before the reference administration, the patient is selected as having the potential to obtain a therapeutic effect with the anti-CD26 antibody.

6. The method according to claim 1, wherein
when the patient is a patient to whom the anti-CD26 antibody is administered with a frequency of once every two weeks, and
the measurement date is Day 29 from the reference administration, and
the level of soluble CD26 in the serum of the patient on the measurement date is less than 62.3% of the level of soluble CD26 in the serum of the patient before the reference administration, the patient is selected as having the potential to obtain a therapeutic effect with the anti-CD26 antibody.

7. The method of claim 5, wherein the patient is male.

8. The method according to claim 1, wherein
when the patient is a patient to whom the anti-CD26 antibody is administered with a frequency of once a week, and
the measurement date is Day 2 to Day 8 from the reference administration, and
the level of soluble CD26 in the serum of the patient on the measurement date is less than 50% of the level of soluble CD26 in the serum of the patient before the reference administration, the patient is selected as having the potential to obtain a therapeutic effect with the anti-CD26 antibody.

9. The method according to claim 8, wherein when the level of soluble CD26 in the serum of the patient on the measurement date is less than 49% of the level of soluble CD26 in the serum of the patient before the reference administration, the patient is selected as having the potential to obtain a therapeutic effect with the anti-CD26 antibody.

10. The method according to claim 1, wherein
when the patient is a patient to whom the anti-CD26 antibody is administered with a frequency of once a week, and
the measurement date is on or after Day 15 from reference administration, and
the level of soluble CD26 in the serum of the patient on the measurement date is less than 30% of the level of soluble CD26 in the serum of the patient before the reference administration, the patient is selected as having the potential to obtain a therapeutic effect with the anti-CD26 antibody.

11. The method of claim 10, wherein when the level of soluble CD26 in the serum of the patient on the measurement date is less than 26% of the level of soluble CD26 in the serum of the patient before the reference administration, the patient is selected as having the potential to obtain a therapeutic effect with the anti-CD26 antibody.

12. The method of claim 8, wherein the patient is a patient to whom 6 mg/kg of the anti-CD26 antibody is administered.

13-46. (canceled)

47. The method according to claim 1, wherein the patient is a patient to whom the anti-CD26 antibody is administered at a frequency of once every two weeks or once a week.

48. The method of claim 1, wherein the anti-CD26 antibody is YS110.

49. The method of claim 1, wherein the cancer is malignant mesothelioma.
